(19) United States
(12) Patent Application Publication
(10) Pub. No.: US 2025/0032487 A1
(43) Pub. Date: Jan. 30, 2025

(54) DISSOLUTION TEST

(71) Applicant: JANSSEN SCIENCES IRELAND UNLIMITED COMPANY, Co. Cork (IE)

(72) Inventors: Johannes Jan MOES, Goes (NL); Inge PIRLET, Geel (BE); Roy Johan E. DE MAESSCHALCK, Turnhout (BE)

(21) Appl. No.: 18/710,770

(22) PCT Filed: Nov. 16, 2022
(86) PCT No.: PCT/EP2022/082130

§ 371 Date: May 16, 2024

Related U.S. Application Data
(63) Continuation of application No. PCT/US2021/072453, filed on Nov. 17, 2021.
(60) Provisional application No. 63/342,834, filed on May 17, 2022.

(30) Foreign Application Priority Data
May 17, 2022 (EP) 22173914.7

(51) Int. Cl. A61K 31/505; G01N 30/02; G01N 30/74; G01N 33/15

(52) U.S. Cl. CPC A61K 31/505; G01N 30/74; G01N 33/15; G01N 2030/027

(57) ABSTRACT

The present invention relates to testing samples comprising rilpivirine or a pharmaceutically acceptable salt thereof in the form of micro- or nanoparticles, such as suspensions, and measuring the dissolution of the rilpivirine or a pharmaceutically acceptable salt thereof in an aqueous medium. The present invention also relates to quality control testing of said samples and to releasing batches comprising said samples for pharmaceutical use. The present invention also relates to a medium for use in dissolution testing.

### TECHNICAL FIELD

The present invention relates to testing samples comprising rilpivirine or a pharmaceutically acceptable salt thereof in the form of micro- or nanoparticles, such as suspensions, and measuring the dissolution of the rilpivirine or a pharmaceutically acceptable salt thereof in an aqueous medium. The present invention also relates to quality control testing of said samples and to releasing batches comprising said samples for pharmaceutical use. The present invention also relates to a medium for use in dissolution testing.

### BACKGROUND AND RELATED ART

The treatment of human immunodeficiency virus (HIV) infection, known as the cause of the acquired immunodeficiency syndrome (AIDS), remains a major medical challenge. Rilpivirine is an anti-retroviral of the non-nucleoside reverse transcriptase inhibitor (NNRTI) class that is used for the treatment of HIV infection. Rilpivirine is a second-generation NNRTI with higher potency and a reduced side effect profile compared with older NNRTIs. Rilpivirine, its pharmacological activity, as well as a number of procedures for its preparation have been described in WO 03/16306. Rilpivirine has been approved for the treatment of HIV infection and is commercially available as a single agent tablet (EDURANT®) containing 25 mg of rilpivirine base equivalent per tablet for once-daily oral administration as well as single tablet regimens for once-daily oral administration (COMPLERA®, ODEFSEY®, JULUCA®).

WO2007/147882 discloses intramuscular or subcutaneous injection of a therapeutically effective amount of rilpivirine in micro- or nanoparticle form, having a surface modifier adsorbed to the surface thereof; and a pharmaceutically acceptable aqueous carrier; wherein the rilpivirine active ingredient is suspended in the pharmaceutically acceptable aqueous carrier. A prolonged release suspension for injection of rilpivirine for administration in combination with a prolonged release suspension for injection of cabotegravir has been approved as CABENUVA® in e.g. the US and Canada and as REKAMBYS® in e.g. the EU. These are the first anti-retrovirals to be provided in a long-acting injectable formulation for administration at intervals of greater than one day.

An issue encountered within pharmaceutical development is the need to control the level of drug available in the systemic circulation to remain within its desired therapeutic window. Drug levels outside the therapeutic window could potentially lead to insufficient or lack of efficacy in case of too low drug levels, or conversely, too high drug levels could potentially produce unwanted adverse events to the patient. The systemic drug levels are a result of the absorption, distribution, metabolism, and excretion of the drug substance. The absorption rate is influenced by the release rate of the drug product. In order to assist with drug level control, dissolution testing is a standardized method for measuring drug release from a given dosage form. Dissolution testing should be both robust and reproducible, with the ability to detect any key changes in product performance, e.g. discriminate between different formulations, manufacturing process parameters, changes during stability, and/or batches. The dissolution test method is also used to guide formulation development and select formulations and batches for clinical trials. A reliable dissolution test is thus a key tool during several stages of pharmaceutical development. Also during pharmaceutical production and quality control, dissolution testing is a valuable tool. The results obtained by dissolution testing can be employed to detect potential variances that may occur during manufacturing as well as ensure batch-to-batch reproducibility, or to release batches for further manufacture into an approved product.

The conditions used for dissolution testing, which is an in vitro technique, are typically chosen to mimic as closely as possible the conditions in vivo in which the drug is released from its dosage form. This is one way for the results of the in vitro test to be considered biorelevant. The conditions include the temperature of the medium used in the dissolution test. Another variable of a dissolution test is the nature of the medium in which the drug substance is dissolved, e.g. its composition and pH. Several methods for dissolution testing of dosage forms are described in compendia such as the US and European pharmacopeia. Also the US FDA publishes methods for dissolution testing of drugs approved by the FDA, specifying conditions and the medium. Dissolution tests for dosage forms comprising rilpivirine published by the FDA (e.g. at https://www.accessdata.fda.gov/scripts/cder/dissolution/) include the following (where no temperature is recited it is generally understood that physiological temperature is chosen, e.g. 37° C. for oral administration):

| | | | | | | Recommended |
|---|---|---|---|---|---|---|
| | | | | | | Sampling |
| | Dosage | USP | Speed | | Volume | Times |
| Drug Name | Form | Apparatus | (RPMs) | Medium | (mL) | (minutes) |
| Dolutegravir | Tablet | II (Paddle) | 75 | 1.0% Tween | 900 | 10, 15, 20, 30, |
| Na/Rilpivirine | | | | 20 in 0.01 M | | 45 and 60 |
| HCl | | | | HCl, pH 2.0 | | |
| Emtricitabine/ | Tablet | II (Paddle) | 75 | Rilpivirine | RPV: | 5, 10, 15, 20, |
| Rilpivirine | | | | (RPV): 0.5% | 1000 mL; | 30 and 45 |
| HCl/Tenofovir | | | | Polysorbate | ETC and | |
| Alafenamide | | | | 20 in 0.01N | TAF: 500 | |
| Fumarate | | | | HCl; | mL | |
| | | | | Emtricitabine | | |
| | | | | (ETC) and | | |
| | | | | Tenofovir | | |
| | | | | alafenamide | | |
| | | | | (TAF): 50 | | |
| | | | | mM Sodium | | |
| | | | | Citrate, pH | | |
| | | | | 5.5 | | |
| Emtricitabine/ | Tablet | II (Paddle) | 75 | 0.5%(w/w) | 1000 | Emtricitabine |
| Rilpivirine | | with sinker | | polysorbate | | and Tenofovir: |
| HCl/Tenofovir | | | | 20 in 0.01N | | 5, 10, 15, 20 |
| Disoproxil | | | | HCl (pH 2.0) | | and 30; |
| Fumarate | | | | | | Rilpivirine: 10, |
| | | | | | | 20, 30, 45, 60, |
| | | | | | | 75, 90 and 120 |
| Rilpivirine | Tablet | II (Paddle) | 75 | 0.5% | 900 | 10, 20, 30, 45 |
| HCl | | | | Polysorbate | | and 60 |
| | | | | 20 in 0.01N | | |
| | | | | HCl (pH = 2.0) | | |

Although dissolution testing was initially developed for immediate release oral solid dosage forms, its use has been extended to formulations which have controlled and modified drug release profiles. It is, however, difficult to design a suitable dissolution test for a dosage form with prolonged release intended for intramuscular or subcutaneous injection, in particular due to the prolonged release, and there is no established strategy for solving this problem. Therefore, there remains a need to develop a reliable dissolution test for prolonged release dosage forms of rilpivirine, in particular for prolonged-release rilpivirine in the form of nano- or microparticles for injection.

### SUMMARY OF THE INVENTION

Accordingly, in a first aspect, the invention provides a method of testing a sample of rilpivirine or a pharmaceutically acceptable salt thereof, wherein the sample comprises rilpivirine or a pharmaceutically acceptable salt thereof in the form of micro- or nanoparticles, the method comprising:
dispersing the sample into an aqueous medium, wherein the aqueous medium:
   comprises a surfactant, and
   is maintained at a temperature of 2-15° C.; and
measuring the dissolution of the rilpivirine or a pharmaceutically acceptable salt thereof in the aqueous medium.

In a second aspect, the invention provides a method of quality control testing a sample of rilpivirine or a pharmaceutically acceptable salt thereof, wherein the sample comprises rilpivirine or a pharmaceutically acceptable salt thereof in the form of micro- or nanoparticles, the method comprising:
performing the method of the first aspect on the sample; and
determining based on the measured dissolution of the rilpivirine or a pharmaceutically acceptable salt thereof in the aqueous medium whether the sample has passed the quality control test.

In a third aspect, the invention provides a method of releasing a batch of rilpivirine or a pharmaceutically acceptable salt thereof for pharmaceutical use, the method comprising: providing a batch of rilpivirine or a pharmaceutically acceptable salt thereof in the form of micro- or nanoparticles, optionally in suspension;
performing the method of quality control of the second aspect on a sample taken from the batch; and
if the sample passes the quality control test, releasing the batch for pharmaceutical use.

In a fourth aspect, the invention provides an aqueous medium for use in dissolution testing, the aqueous medium:
comprising 4-8% w/v, or 5.5-6.5% w/v, or 5.94-6.06% w/v of a surfactant, in particular a non-ionic surfactant such as polysorbate 20;
comprising a buffer, such as 0.05 M sodium phosphate buffer; and
having a pH of 6-8, 7-8, 7.2-7.8, or 7.3-7.5.

### BRIEF DESCRIPTION OF THE FIGURES

The invention will be described, by way of example only, with reference to the accompanying figures.

FIG. 1: Dissolution studies with rilpivirine suspensions of varying particle size

FIG. 2: Dissolution studies with rilpivirine suspensions of varying particle size

FIG. 3: Dissolution studies with rilpivirine suspensions of varying particle size

FIG. 4: Dissolution studies of rilpivirine suspensions at varying temperature

FIG. 5: Dissolution studies of rilpivirine suspensions at varying surfactant concentrations

FIG. 6: Equilibrium solubility of rilpivirine at varying surfactant concentrations

FIG. 7: Dissolution studies of rilpivirine suspensions after varying storage conditions

These figures are explained further in the “Examples” section.

### DISCLOSURE OF THE INVENTION

This application has been drafted in sections to aid readability. However, this does not mean that each section is to be read in isolation. To the contrary, unless otherwise specified, each section is to be read with cross-referencing to the other sections, i.e. taking the entire application as a whole. No artificial separation of embodiments is intended, unless explicitly stated.

Thus, all of the embodiments described herein relating to the first aspect of the invention apply equally to, i.e. are also disclosed in relation to/combination with aspects two to four herein. For example, the features of the aqueous medium described in connection with the first aspect apply to the second, third, and fourth aspects. The features of the sample comprising rilpivirine or a pharmaceutically acceptable salt thereof in the form of micro- or nanoparticles apply to the first, second, and third aspects.

### DETAILED DESCRIPTION OF THE INVENTION

### Dissolution Test

The method of the first aspect of the invention is unusual in that it measures the dissolution of the rilpivirine or a pharmaceutically acceptable salt thereof at temperatures significantly below physiological temperatures. Physiological temperatures are typically chosen for dissolution testing as they may render the in vitro test representative of the behaviour of the drug substance in vivo. The typical temperature for measuring the dissolution of an oral formulation form is thus 37° C. However, the inventors have surprisingly found that the dissolution of rilpivirine or a pharmaceutically acceptable salt thereof in the form of micro- or nanoparticles can be measured at the low temperatures of 2-15° C. which was found to improve the discriminating abilities of the method, in particular enabling samples of different particle size to be discriminated. Lowering the temperature was also found to slow down the dissolution, thus allowing the method to evaluate the potential for burst release of the drug substance from the dosage form. Given the discriminative properties of the test, it can be considered to provide biorelevant results. Moreover, the method was found to enable, over a practical timescale suitable for laboratory testing purposes, the in vitro studies of the dissolution of rilpivirine or a pharmaceutically acceptable salt thereof in the form of micro- or nanoparticles intended for prolonged release. Due to these advantages, the invention also provides in a second aspect an improved method of quality control testing a sample of rilpivirine or a pharmaceutically acceptable salt thereof in the form of micro- or nanoparticles. In a third aspect the invention provides an improved method of releasing a batch of rilpivirine or a pharmaceutically acceptable salt thereof for pharmaceutical use.

In an embodiment, the aqueous medium is maintained at a temperature of 3-10° C., or 4-6° C., preferably 4.5-5.5° C., in particular 5° C. Using a temperature within a narrow range, e.g. a set temperature ±0.5° C., for each iteration of the dissolution method may improve the robustness of the method.

Preferably the sample or the formulation to be tested is a suspension of micro- or nanoparticles of rilpivirine or a pharmaceutically acceptable salt thereof in a pharmaceutically acceptable carrier, such as a pharmaceutically acceptable aqueous carrier. Suspensions are described further below. When the formulation or sample is a suspension, preferably it is fully resuspended and homogenized prior to the step of dispersing the sample into the aqueous medium. The homogenization may comprise mechanical homogenization, for example using a vortex mixer; may comprise manual homogenization, for example shaking by hand; and may comprise both mechanical homogenization and manual homogenization. A homogenization protocol may be established to be used for each iteration of the dissolution test to eliminate any potential dependence of the results on the homogenization conditions. For example, a homogenization protocol may require homogenizing a vial containing the sample using a vortex mixer for at least 15 seconds followed by manually shaking the vial horizontally 30 times over approximately 25 cm within approximately 10 seconds.

The method is preferably not performed at sink conditions. Sink conditions are defined as conditions wherein the equilibrium solubility of rilpivirine or a pharmaceutically acceptable salt thereof in the aqueous medium is at least 3 times higher than the concentration that would be obtained if all the rilpivirine or a pharmaceutically acceptable salt thereof from the sample dissolves in the aqueous medium. It will be understood that equilibrium solubility refers to the concentration of a substance in a solvent when that substance is in dynamic equilibrium between the solid state and the dissolved state in the solvent. Sink conditions are usually deemed to be essential in dissolution testing methods to allow the dissolution rate to be consistently measured: otherwise, when the concentration of the dissolved drug substance in the aqueous medium approaches the equilibrium solubility, the dissolution rate is believed to reduce in such a way as to affect the reproducibility of the test results. Surprisingly, the inventors have found that the method of the invention may be performed not at sink conditions while still providing excellent reproducibility and discriminating abilities; the discriminating abilities may be better when the method is performed not at sink conditions than when it is performed at sink conditions.

Preferably, the concentration that would be obtained if all the rilpivirine or a pharmaceutically acceptable salt thereof from the sample dissolves in the aqueous medium is equal to or lower than the equilibrium solubility of rilpivirine or a pharmaceutically acceptable salt thereof in the aqueous medium. In this way, the dissolution of all of the rilpivirine or a pharmaceutically acceptable salt thereof from the sample can be measured, for example using an infinity point as discussed further below.

In an embodiment, the concentration that would be obtained if all the rilpivirine or a pharmaceutically acceptable salt thereof from the sample dissolves in the aqueous medium is equal to or higher than the equilibrium solubility of rilpivirine or a pharmaceutically acceptable salt thereof in the aqueous medium. In this way, not all rilpivirine or a pharmaceutically acceptable salt thereof will be dissolved from the sample and not all of the rilpivirine or a pharmaceutically acceptable salt thereof from the sample can be measured, for instance at least 80% of rilpivirine or a pharmaceutically acceptable salt thereof from the sample will be dissolved, or at least 85% of rilpivirine or a pharmaceutically acceptable salt thereof from the sample will be dissolved, or at least 90% of rilpivirine or a pharmaceutically acceptable salt thereof from the sample will be dissolved, or at least 95% of rilpivirine or a pharmaceutically acceptable salt thereof from the sample will be dissolved.

Whether a system, e.g. a specific sample in combination with a specific aqueous medium, is at sink conditions can be controlled by varying parameters which affect the equilibrium concentration, e.g. the temperature, pH, and/or surfactant concentration of the aqueous medium.

Whether a system, e.g. a specific sample in combination with a specific aqueous medium, is at sink conditions can be controlled by varying the concentration that would be obtained if all the rilpivirine or a pharmaceutically acceptable salt thereof from the sample dissolves in the medium, e.g. varying the amount of rilpivirine or a pharmaceutically acceptable salt thereof in the sample, and/or varying the volume of the medium, and/or varying the volume or weight of the sample. The sample may contain 10-30 mg, or 16-20 mg, or 17.1-18.9 mg rilpivirine or a pharmaceutically acceptable salt thereof. The volume of the aqueous medium may be 500-1500 mL, or 700-1, 100 mL, or about 900 mL. When all the rilpivirine or a pharmaceutically acceptable salt thereof from the sample dissolves in the aqueous medium, the concentration of rilpivirine or a pharmaceutically acceptable salt thereof in the aqueous media may be about 0.015-0.025 mg/mL, or about 0.019-0.021, or about 0.020 mg/mL. These concentrations preferably represent conditions which are not sink conditions.

The aqueous medium comprises a surfactant. The surfactant aids the dissolution of the rilpivirine or a pharmaceutically acceptable salt thereof in the aqueous medium. The surfactant should be selected such that it does not crystallise at the low temperature used for the method. The surfactant may be a non-ionic surfactant such as a polysorbate (available as Tween™ surfactants); a poly(alkylene-oxide) block copolymer such as poly(ethylene oxide)-poly(propylene oxide)-poly(ethylene oxide) (available as Pluronic™ surfactants), poly(propylene oxide)-poly(ethylene oxide)-poly(propylene oxide) (available as Pluronic R™ surfactants), poly(ethylene oxide)-poly(butylene oxide)-poly(ethylene oxide), poly(butylene oxide)-poly(ethylene oxide), and tetrafunctional poly(alkylene-oxide) block copolymers (available as Tetronic™ surfactants); an oligomeric alkyl-ethylene oxide (available as Brij™ or Tergitol™ surfactants); an alkyl-phenol-polyethylene (available as Triton™ surfactants); and mixtures thereof. In an embodiment, the surfactant may be a non-ionic surfactant such as a polysorbate (available as Tween™ surfactants); an oligomeric alkyl-ethylene oxide (available as Brij™ or Tergitol™ surfactants). Preferably, the surfactant is polysorbate 20.

In an embodiment, the surfactant is a sorbitan ester, e.g. sorbitan oleate (available as Span™ surfactants).

The concentration of the surfactant may be controlled to further improve the discriminating properties of the dissolution method. For instance, the surfactant concentration may be controlled to affect the dissolution profile, and hence the performance of the method. A suitable performing method is able to detect a potential burst release (initial release of the reference (first time point to measure the dissolution is preferably measured between 1 or 5 minutes after start of the experiment, e.g. at 1, 2, 3, 4 or 5 minutes) is preferably below 10% dissolved, or below 20% dissolved, or below 25% dissolved or below 30% dissolved), characterize the release profile (sufficient time points between 20% and 65% dissolved), and detect final release above 50%, or 60%, or 70%, or 80%, or 90% dissolved, preferably 100% dissolved. The performance of each method can be defined by calculating the difference between the lowest and highest % dissolved in the dissolution profile, i.e. the delta % dissolved. For instance, the delta % dissolved of the 6% polysorbate 20 method is approximately 80%. The higher the % dissolved, the higher the ability of the method to discriminate between different particle sizes of rilpivirine. In an embodiment, the delta % dissolved is at least 40%, at least 50%, at least 60%, at least 70%, at least 80%. Likewise, one could optimize the method by controlling the surfactant concentrations to increase the delta % dissolved. Accordingly, the surfactant, e.g. polysorbate 20, may be present in the aqueous medium at a concentration of 2-8% w/v, or 4-8% w/v, or 5.5-6.5% w/v, or 5.94-6.06% w/v, or 5% w/v, or 5.5% w/v or 6% w/v. Using a concentration within a narrow range, e.g. a set concentration ±1%, for each iteration of the dissolution method may improve the robustness of the method.

The aqueous medium may contain a buffer. It has been found that a variety of buffers may be used while maintaining the discriminating properties of the method. Suitable buffers include phosphate buffer, citrate buffer, citrate-phosphate buffer (e.g. Mcllvaine buffer), tris(hydroxymethyl) aminomethane buffer, borate buffer, phthalate buffer, acetate buffer, and mixtures thereof. A preferred buffer is 0.05 M sodium phosphate buffer.

In an embodiment, the aqueous medium contains a pH adjusting agent, e.g. sodium hydroxide.

One of the factors that may be controlled to influence the solubility of the rilpivirine is the pH of the aqueous medium. The aqueous medium may have a pH of 6-8, 7-8, 7.2-7.8, or 7.3-7.5. Using a pH within a narrow range, e.g. a set pH ±0.1, for each iteration of the dissolution method may improve the robustness of the method. The choice of a pH in the recited range for measuring the dissolution of rilpivirine is unusual. For instance, each of the dissolution tests for dosage forms comprising rilpivirine published by the US FDA involves an aqueous medium at pH 2.0.

When multiple samples are to be tested, preferably the method comprises a first iteration of the dissolution test on a first sample and a second iteration of the dissolution test on a second sample, wherein the concentration of the surfactant in the aqueous medium in the second iteration is maintained within ±1% of the concentration of surfactant in the aqueous medium in the first iteration, the temperature of the aqueous medium in the second iteration is maintained within ±0.5° C. of the temperature of the aqueous medium in the first iteration, and the pH of the aqueous medium in the second iteration is maintained within ±0.1 of the pH of the aqueous medium in the first iteration. In this way the results of the first iteration and the second iteration can be directly compared.

Most preferably, the aqueous medium comprises 5.94-6.06% w/v polysorbate 20; comprises 0.05 M sodium phosphate buffer; has a pH of 7.3-7.5; and is maintained at a temperature of 4.5-5.5° C.

The dissolution method may be performed in any suitable apparatus, such as standard dissolution instrumentation described in the pharmacopeia, for example USP 42-NF 37 2019. Dispersing the sample of rilpivirine or a pharmaceutically acceptable salt thereof in the form of micro- or nanoparticles into the aqueous medium typically comprises agitation. For example, a paddle apparatus may be used, in particular a USP type 2 apparatus. The rotation speed of the apparatus is typically 10-100 rpm, or 25-75 rpm, or about 50 rpm.

In vitro, the dissolution of a drug is generally monitored for a time period which is similar to the time needed for in vivo drug release. Accordingly, this would mean monitoring the dissolution over several weeks or several months for a sample of rilpivirine or a pharmaceutically acceptable salt thereof in the form of micro- or nanoparticles intended for administration by intramuscular or subcutaneous injection for the long-term treatment of HIV infection, or for the long-term prevention of HIV infection, e.g. a sample of a prolonged release injectable rilpivirine suspension. Long-term treatment of HIV infection or long-term prevention of HIV infection in a subject at risk of being infected by HIV can be understood as the treatment of HIV infection or the prevention of HIV infection in a subject at risk of being infected by HIV wherein the rilpivirine or a pharmaceutically acceptable salt thereof in the form of micro- or nanoparticles, optionally in suspension, is administered subcutaneously or intramuscularly intermittently at a time interval in the range of 1 week to 2 years, or 2 weeks to 1 year, or 1 month to 6 months, or about 1 month, or about 2 months, or about 3 months, or about 4 months, or about 5 months, or about 6 months. However, this can be impractical for quality control purposes and for development purposes. Accordingly, the measurement of the dissolution of the rilpivirine or a pharmaceutically acceptable salt thereof in the aqueous medium may be performed over 24 hours, or 4-8 hours, or 5-7 hours, or about 6 hours. The inventors have found that the in vitro method provides a result that is biorelevant due to its discriminative properties despite the significant difference between the in vitro monitoring period (in the order of hours) and the in vivo drug release period (in the order of weeks or months).

The dissolution test may be operated such that at least 80% or at least 85% of the rilpivirine or a pharmaceutically acceptable salt thereof from the sample has dissolved in the aqueous medium after about 6 hours. In this way, the dissolution of a sufficient amount of the sample to provide robust results is determined over a practical timescale.

The dissolution test may provide a measured dissolution profile of the rilpivirine or a pharmaceutically acceptable salt thereof in the aqueous medium characterized by one or more, optionally all, of features (a)-(l):
(a) about 14% of the rilpivirine or a pharmaceutically acceptable salt thereof dissolves after about 5 minutes;
(b) about 25% of the rilpivirine or a pharmaceutically acceptable salt thereof dissolves after about 10 minutes;
(c) about 34% of the rilpivirine or a pharmaceutically acceptable salt thereof dissolves after about 15 minutes;
(d) about 52% of the rilpivirine or a pharmaceutically acceptable salt thereof dissolves after about 30 minutes;
(e) about 62% of the rilpivirine or a pharmaceutically acceptable salt thereof dissolves after about 45 minutes;
(f) about 69% of the rilpivirine or a pharmaceutically acceptable salt thereof dissolves after about 60 minutes;
(g) about 77% of the rilpivirine or a pharmaceutically acceptable salt thereof dissolves after about 90 minutes;
(h) about 82% of the rilpivirine or a pharmaceutically acceptable salt thereof dissolves after about 120 minutes;
(i) about 88% of the rilpivirine or a pharmaceutically acceptable salt thereof dissolves after about 180 minutes;
(j) about 91% of the rilpivirine or a pharmaceutically acceptable salt thereof dissolves after about 240 minutes;
(k) about 94% of the rilpivirine or a pharmaceutically acceptable salt thereof dissolves after about 360 minutes.

For example, features (a), (d), and (j) may be present; or (a), (c), (e), and (i) may be present; or all of (a)-(k) may be present.

The dissolution test may provide a measured dissolution profile of the rilpivirine or a pharmaceutically acceptable salt thereof in the aqueous medium characterized by one or more, optionally all, of features (i)-(vi):
(i) at 5 minutes, ≤30% of the rilpivirine or a pharmaceutically acceptable salt thereof dissolves;
(ii) at 10 minutes, 10-40% of the rilpivirine or a pharmaceutically acceptable salt thereof dissolves;
(iii) at 30 minutes, 39-59% of the rilpivirine or a pharmaceutically acceptable salt thereof dissolves;
(iv) at 45 minutes, 45-75% of the rilpivirine or a pharmaceutically acceptable salt thereof dissolves;
(v) at 90 minutes, 64-84% of the rilpivirine or a pharmaceutically acceptable salt thereof dissolves;
(vi) at 360 minutes, ≥80% of the rilpivirine or a pharmaceutically acceptable salt thereof dissolves.

For example, features (ii), (iv), and (vi) may be present; or preferably features (i), (iii), (v), and (vi) may be present.

The dissolution test may comprise measuring the dissolution of the rilpivirine or a pharmaceutically acceptable salt thereof in the aqueous medium at an infinity point wherein at least about 80%, at least about 85%, at least about 90%, at least about 95%, or preferably about 100% (i.e., about all) of the rilpivirine or a pharmaceutically acceptable salt thereof from the sample dissolves in the aqueous medium. The infinity point may be achieved by increasing the temperature of the aqueous medium from the initial temperature (e.g. 2-15, 3-10, 4-6, or 4.5-5.5° C.) to room temperature (e.g. about 22° C.) or above such as about 37° C., and optionally maintaining the aqueous medium at the increased temperature for about 1 hour. For example, in an embodiment, the dissolution test may comprise measuring the dissolution of the rilpivirine or a pharmaceutically acceptable salt thereof in the aqueous medium at 2-15° C. as a function of time, optionally over a period of 3-8 hours, or 4-8 hours, or 5-7 hours, or about 6 hours, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium. For example, in an embodiment, the dissolution test may comprise measuring the dissolution of the rilpivirine or a pharmaceutically acceptable salt thereof in the aqueous medium at 2-15° C., until at least about 40%, at least about 50%, at least about 60%, at least about 70%, at least about 80%, at least about 85%, or at least about 90%, or at least about 95% of the rilpivirine or a pharmaceutically acceptable salt thereof from the sample dissolves, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium. For example, in an embodiment, the dissolution test may comprise measuring the dissolution of the rilpivirine or a pharmaceutically acceptable salt thereof in the aqueous medium at 2-15° C., until about 40%, about 50%, about 60%, about 70%, about 80%, about 85%, or about 90%, or about 95% of the rilpivirine or a pharmaceutically acceptable salt thereof from the sample dissolves, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium. For example, in an embodiment, the dissolution test may comprise measuring the dissolution of the rilpivirine or a pharmaceutically acceptable salt thereof in the aqueous medium at 2-15° C. as a function of time, over a period of 3-8 hours, or 4-8 hours, or 5-7 hours, or about 6 hours, and until at least about 40%, at least about 50%, at least about 60%, at least about 70%, at least about 80%, at least about 85%, or at least about 90%, or at least about 95% of the rilpivirine or a pharmaceutically acceptable salt thereof from the sample dissolves, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium. For example, in an embodiment, the dissolution test may comprise measuring the dissolution of the rilpivirine or a pharmaceutically acceptable salt thereof in the aqueous medium at 2-15° C. as a function of time, over a period of 3-8 hours, or 4-8 hours, or 5-7 hours, or about 6 hours, and until about 40%, about 50%, about 60%, about 70%, about 80%, about 85%, or about 90%, or about 95% of the rilpivirine or a pharmaceutically acceptable salt thereof from the sample dissolves, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium. For example, in an embodiment, the dissolution test may comprise measuring the dissolution of the rilpivirine or a pharmaceutically acceptable salt thereof in the aqueous medium at 2-15° C. as a function of time, over a period of 3-8 hours, or 4-8 hours, or 5-7 hours, or about 6 hours, and until at least about 80%, at least about 85%, or at least about 90%, or at least about 95% of the rilpivirine or a pharmaceutically acceptable salt thereof from the sample dissolves, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium. For example, in an embodiment, the dissolution test may comprise measuring the dissolution of the rilpivirine or a pharmaceutically acceptable salt thereof in the aqueous medium at 2-15° C. as a function of time, over a period of 3-8 hours, or 4-8 hours, or 5-7 hours, or about 6 hours, and until about 80%, about 85%, or about 90%, or about 95% of the rilpivirine or a pharmaceutically acceptable salt thereof from the sample dissolves, and comprising a subsequent step of increasing the temperature of the aqueous medium to room temperature (e.g. about 22° C.) or above such as about 37° C., maintaining the aqueous medium at the increased temperature for about 1 hour, and measuring the dissolution of the rilpivirine or a pharmaceutically acceptable salt thereof in the aqueous medium.

Measuring the dissolution of the rilpivirine or a pharmaceutically acceptable salt thereof in the aqueous medium can be readily achieved by removing an aliquot from the medium, optionally filtering the aliquot, and measuring the amount of rilpivirine or a pharmaceutically acceptable salt thereof dissolved in the aliquot. The filtering removes undissolved rilpivirine particles. It has been found that a filter, e.g. a syringe filter, with a pore size of 0.1 μm, e.g. a regenerated cellulose or polyvinylidene difluoride (PVDF) membrane, is suitable. If the aliquot is filtered, typically a new filter is used for each aliquot to avoid possible contamination. Alternatively, the aliquot could be centrifuged, cooled, and/or diluted before measuring the amount of rilpivirine or a pharmaceutically acceptable salt thereof dissolved in the aliquot. When more than one aliquot has been taken from the vessel the % dissolved should be corrected to reflect the removal of rilpivirine or a pharmaceutically acceptable salt thereof and volume of dissolution medium.

The quantity of rilpivirine or a pharmaceutically acceptable salt thereof present in the aliquots may be determined by standard techniques such as high performance liquid chromatography (HPLC), in particular gradient ultra-high performance liquid chromatography (UHPLC) with UV detection.

Measuring the dissolution of the rilpivirine or a pharmaceutically acceptable salt thereof in the aqueous medium can also be achieved without removal of an aliquot using in-line spectroscopy techniques such as in-line UV spectroscopy.

In the fourth aspect the invention provides an aqueous medium for use in dissolution testing, the medium comprising 4-8% w/v, or 5.5-6.5% w/v, or 5.94-6.06% w/v of a surfactant, e.g. a non-ionic surfactant such as polysorbate 20; comprising a buffer, such as 0.05 M sodium phosphate buffer; and having a pH of 6-8, 7-8, 7.2-7.8, or 7.3-7.5. This represents a particularly effective aqueous medium for use in the dissolution test method of the first aspect. Preferably, the aqueous medium comprises 5.94-6.06% w/v of polysorbate 20; comprises a buffer, such as 0.05 M sodium phosphate buffer; and has a pH of 7.3-7.5. The aqueous medium may be maintained at a temperature of 2-15, 3-10, 4-6, or preferably 4.5-5.5° C. The aqueous medium may comprise dissolved rilpivirine or a pharmaceutically acceptable salt thereof, e.g. present from the dissolution testing.

### Quality Control

In the second aspect, the results of the dissolution test are used for quality control testing of the sample of rilpivirine or a pharmaceutically acceptable salt thereof. For instance, as shown in the examples, the test of the first aspect discriminates between different particle size distributions. Therefore, in the second aspect the measured dissolution of the rilpivirine or a pharmaceutically acceptable salt thereof in the aqueous medium is preferably used to determine whether the sample comprising rilpivirine or a pharmaceutically acceptable salt thereof in the form of micro- or nanoparticles meets a specified particle size distribution, e.g. a specified Dᵥ50, or a specified Dᵥ90, or a specified Dᵥ10, or a specified Dᵥ10, Dᵥ50 and Dᵥ90. Determining whether a specified particle size distribution has been achieved is an important step in the manufacture of certain formulations of rilpivirine or a pharmaceutically acceptable salt thereof for pharmaceutical use. Moreover, some agglomeration may occur on storing rilpivirine or a pharmaceutically acceptable salt thereof in the form of micro- or nanoparticles, altering the particle size distribution. Therefore, the measured dissolution of the rilpivirine in the medium may be used to determine whether rilpivirine or a pharmaceutically acceptable salt thereof in the form of micro- or nanoparticles which has been stored for a period of time retained its particle size distribution.

Determining whether the sample has passed the quality control test may be achieved by comparing the measured dissolution of the rilpivirine or a pharmaceutically acceptable salt thereof in the aqueous medium with one or more reference values of the dissolution of a reference sample of rilpivirine or a pharmaceutically acceptable salt thereof in the form of micro- or nanoparticles and determining, based on the comparison, whether the sample has passed the quality control test. For example, the determining may comprise comparing the measured dissolution with one or more reference values at a single time point, or at least two time points, or preferably at least three time points.

The determining may comprise comparing the measured dissolution with one or more reference values, wherein the sample is determined to pass the quality control test if the measured dissolution meets one or more, optionally all, of reference values (i)-(vi):
(i) at 5 minutes, ≤30% of the rilpivirine or a pharmaceutically acceptable salt thereof dissolves;
(ii) at 10 minutes, 10-40% of the rilpivirine or a pharmaceutically acceptable salt thereof dissolves;
(iii) at 30 minutes, 39-59% of the rilpivirine or a pharmaceutically acceptable salt thereof dissolves;
(iv) at 45 minutes, 45-75% of the rilpivirine or a pharmaceutically acceptable salt thereof dissolves;
(v) at 90 minutes, 64-84% of the rilpivirine or a pharmaceutically acceptable salt thereof dissolves;
(vi) at 360 minutes, ≥80% of the rilpivirine or a pharmaceutically acceptable salt thereof dissolves.

For example, the sample may be determined to pass the quality control test if the measured dissolution meets values (ii), (iv), and (vi); or preferably meets values (i), (iii), (v), and (vi).

The properties, e.g. the particle size distribution, of the reference sample may have been independently verified by another technique, such as laser diffraction. Preferably the reference values are for the dissolution of the reference sample in an identical medium to the medium into which the sample was dispersed, most preferably wherein the dissolution of the reference sample and the sample were tested using an identical method, since this allows for a direct comparison. In an identical method, the concentration of the surfactant in the aqueous medium when testing the sample is maintained within ±1% of the concentration of surfactant in the aqueous medium when testing the reference sample, the temperature of the aqueous medium when testing the sample is maintained within ±0.5° C. of the temperature of the aqueous medium when testing the reference sample, and the pH of the aqueous medium when testing the sample is maintained within ±0.1 of the pH of the aqueous medium when testing the reference sample.

However, in an aspect, the reference values may be obtained from dissolution in a different medium, provided that the relationship between dissolution in the different mediums is established so that dissolution in the different medium can be correlated to the dissolution of the sample in the chosen medium.

### Release of a Batch for Pharmaceutical Use

In the third aspect, the method of quality control is used to determine whether a batch of rilpivirine or a pharmaceutically acceptable salt thereof in the form of micro- or nanoparticles can be released for pharmaceutical use. For instance, the batch can be released for sale, for supply or for export. Releasing the batch may include providing the batch with documents certifying that the batch is suitable for pharmaceutical use. The batch may be of an approved pharmaceutical product, such as a product approved by the FDA (US Food and Drug Administration), EMA (European Medicines Agency), and/or MHRA (UK Medicines & Healthcare products Regulatory Agency). For example, the batch may be of an NDA drug product, an ANDA drug product, a supplemental New Drug Application drug product, or a 505 (b) (2) drug product.

The pharmaceutical use preferably comprises the treatment of HIV infection or the prevention of HIV infection in a subject at risk of being infected by HIV, most preferably the long-term treatment of HIV infection or the long-term prevention of HIV infection in a subject at risk of being infected by HIV, in particular the treatment of HIV infection or the prevention of HIV infection in a subject at risk of being infected by HIV wherein the rilpivirine or a pharmaceutically acceptable salt thereof in the form of micro- or nanoparticles, optionally in suspension, is administered subcutaneously or intramuscularly intermittently at a time interval in the range of 1 week to 2 years, or 2 weeks to 1 year, or 1 month to 6 months, or about 1 month, or about 2 months, or about 3 months, or about 4 months, or about 5 months, or about 6 months.

The method may be performed as part of a process of manufacturing rilpivirine or a pharmaceutically acceptable salt thereof in the form of micro- or nanoparticles for pharmaceutical use. Therefore, providing the batch may comprise manufacturing the batch. The method may be performed as a means for checking the quality of rilpivirine or a pharmaceutically acceptable salt thereof in the form of micro- or nanoparticles obtained from a supplier. Therefore, providing the batch may comprise obtaining the batch from a supplier. The method may be performed as a means for checking whether a batch of pharmaceutical product that has been stored is in suitable condition for use. Therefore, the batch may have been stored for a period of time before the sample is taken; for example for at least 1 month, 3 months, or 6 months.

It will be understood that the batch refers to a larger amount of rilpivirine or a pharmaceutically acceptable salt thereof in the form of micro- or nanoparticles, and that the sample taken from the batch is a smaller amount considered as representative of the batch. For instance, the batch may comprise at least 100 g, at least 1 kg, or at least 10 kg of rilpivirine or a pharmaceutically acceptable salt thereof in the form of micro- or nanoparticles, optionally in suspension.

Providing the batch encompasses continuous manufacturing processes of rilpivirine or a pharmaceutically acceptable salt thereof in the form of micro- or nanoparticles. Here, the method of quality control may be performed on a sample taken from the product of a continuous manufacturing process, and if the sample passes the quality control test, releasing for pharmaceutical use the batch of the manufacturing process which is contemporaneous with the sample. Samples may be taken from the product of a continuous manufacturing process at set periods of time to confirm whether the process is operating as intended, e.g. that the intended particle size distribution is obtained.

### Rilpivirine

Rilpivirine (4-[[4-[[4-[(1E)-2-cyanoethenyl]-2,6-dimethylphenyl]amino]-2-pyrimidinyl]amino]benzonitrile; TMC278) has the following structural formula:

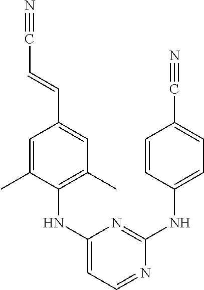 [SMILES: Cc1cc(/C=C/C#N)cc(C)c1Nc1ccnc(Nc2ccc(C#N)cc2)n1]

By “rilpivirine” it is meant rilpivirine having the structural formula shown above, i.e. the free base form.

The rilpivirine or a pharmaceutically acceptable salt thereof is in the form of micro- or nanoparticles, e.g. microparticles or nanoparticles of the rilpivirine or a pharmaceutically acceptable salt thereof in a suspension, in particular micro- or nanoparticles of the rilpivirine or a pharmaceutically acceptable salt thereof suspended in a pharmaceutically acceptable carrier, such as for example a pharmaceutically acceptable aqueous carrier.

Pharmaceutically acceptable salts of rilpivirine means those where the counterion is pharmaceutically acceptable. The pharmaceutically acceptable salts are meant to comprise the therapeutically active non-toxic acid addition salt forms which rilpivirine is able to form. These salt forms can conveniently be obtained by treating rilpivirine with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids.

Preferably the rilpivirine or a pharmaceutically acceptable salt thereof used in the invention is rilpivirine.

The skilled person would understand that the size of the micro- or nanoparticles should be below a maximum size above which administration by subcutaneous or intramuscular injection becomes impaired or even is no longer possible. The maximum size depends for example on the limitations imposed by the needle diameter or by adverse reactions of the body to large particles, or both.

In an embodiment, the rilpivirine or a pharmaceutically acceptable salt thereof is in the form of nanoparticles.

In an embodiment, the rilpivirine or a pharmaceutically acceptable salt thereof is in the form of microparticles.

In an embodiment, the micro- or nanoparticles described herein have a Dᵥ50 particle diameter of less than about 20 μm, or less than about 10 μm, or less than about 2 μm.

Two embodiments having preferred particle sizes for the rilpivirine or pharmaceutically acceptable salt thereof are contemplated herein.

In the first preferred rilpivirine or pharmaceutically acceptable salt thereof particle size embodiment, the particles have a Dᵥ90 of less than or about 2 μm. In this embodiment, the particles may have a Dᵥ90 of from about 100 nm to about 2 μm. In this embodiment, the particles may have a Dᵥ90 of from 200 nm to about 2 μm. In this embodiment, the particles may have a Dᵥ90 of from 300 nm to about 2 μm. In this embodiment, the particles may have a Dᵥ90 of from 400 nm to about 2 μm. In this embodiment, the particles may have a Dᵥ90 of from 500 nm to about 2 μm. Preferably in this embodiment, the particles have a Dᵥ90 of from 500 nm to about 1,600 nm or a Dᵥ90 of from 500 nm to about 1,000 nm.

The term “Dᵥ90” as used herein refers to the diameter below which 90% by volume of the particle population is found. The term “Dᵥ50” as used herein refers to the diameter below which 50% by volume of the particle population is found. The term “Dᵥ10” as used herein refers to the diameter below which 10% by volume of the particle population is found.

In the first preferred rilpivirine or pharmaceutically acceptable salt thereof particle size embodiment, the particles may have a Dᵥ50 of less than or about 1,000 nm. In this embodiment, the particles may have a Dᵥ50 of from about 10 nm to about 1,000 nm. In this embodiment, the particles may have a Dᵥ50 of from about 50 nm to about 700 nm. In this embodiment, the particles may have a Dᵥ50 of from about 100 nm to about 600 nm. In this embodiment, the particles may have a Dᵥ50 of from about 150 nm to about 500 nm. Preferably in this embodiment, the particles have a Dᵥ50 of from about 200 nm to about 500 nm.

In the first preferred rilpivirine or pharmaceutically acceptable salt thereof particle size embodiment, the particles may have a Dᵥ10 of less than or about 500 nm. In this embodiment, the particles may have a Dᵥ10 of from about 10 nm to about 500 nm. In this embodiment, the particles may have a Dᵥ10 of from about 25 nm to about 400 nm. In this embodiment, the particles may have a Dᵥ10 of from about 50 nm to about 300 nm. In this embodiment, the particles may have a Dᵥ10 of from about 50 nm to about 200 nm. Preferably, in this embodiment, the particles have a Dᵥ10 of from about 75 nm to about 200 nm.

Preferably in this embodiment, the rilpivirine or pharmaceutically acceptable salt thereof particles have a Dᵥ90 of from about 500 nm to about 1,600 nm, a Dᵥ50 of from about 200 nm to about 500 nm and a Dᵥ10 of from about 75 nm to about 200 nm.

Alternatively, the rilpivirine or pharmaceutically acceptable salt thereof particles have a Dᵥ90 of from about 500 nm to about 1,000 nm, a Dᵥ50 of from about 200 nm to about 500 nm and a Dᵥ10 of from about 75 nm to about 200 nm.

In the second preferred rilpivirine or pharmaceutically acceptable salt thereof particle size embodiment, the particles may have a Dᵥ90 of from about 1 μm to about 10 μm. In this embodiment, the particles may have a Dᵥ90 of from about 2 μm to about 9 μm. In this embodiment, the particles may have a Dᵥ90 of from about 3 μm to about 8 μm. In this embodiment, the particles may have a Dᵥ90 of from about 3 μm to about 7 μm. Preferably in this embodiment, the particles have a Dᵥ90 of from about 4 μm to about 6 μm.

In the second preferred rilpivirine or pharmaceutically acceptable salt thereof particle size embodiment, the particles have a Dᵥ50 of less than or about 3 μm. In this embodiment, the particles may have a Dᵥ50 of less than about 2.5 μm. In this embodiment, the particles may have a Dᵥ50 of from about 1 μm to about 2.5 μm. In this embodiment, the particles may have a Dᵥ50 of from about 1.2 μm to about 2.2 μm. Preferably in this embodiment, the particles have a Dᵥ50 of from about 1.5 μm to about 2 μm.

In the second preferred rilpivirine or pharmaceutically acceptable salt thereof particle size embodiment, the particles may have a Dᵥ10 of less than or about 1000 nm. In this embodiment, the particles may have a Dᵥ10 of from about 10 nm to about 1000 nm. In this embodiment, the particles may have a Dᵥ10 of from about 100 nm to about 700 nm. In this embodiment, the particles may have a Dᵥ10 of from about 200 nm to about 600 nm. Preferably in this embodiment, the particles have a Dᵥ10 of from about 300 nm to about 500 nm.

Preferably in this embodiment, the rilpivirine or pharmaceutically acceptable salt thereof particles have a Dᵥ90 of from about 4 μm to about 6 μm, a Dᵥ50 of from about 1.5 μm to about 2 μm and a Dᵥ10 of from about 300 nm to about 500 nm.

The Dᵥ10, Dᵥ50 and Dᵥ90 as used herein are determined by routine laser diffraction techniques, e.g. in accordance with ISO 13320:2009.

Laser diffraction relies on the principle that a particle will scatter light at an angle that varies depending on the size the particle and a collection of particles will produce a pattern of scattered light defined by intensity and angle that can be correlated to a particle size distribution. A number of laser diffraction instruments are commercially available for the rapid and reliable determination of particle size distributions. For example, particle size distribution may be measured by the conventional Malvern Mastersizer™ 3000 particle size analyser from Malvern Instruments. The Malvern Mastersizer™ 3000 particle size analyser operates by projecting a helium-neon gas laser beam through a transparent cell containing the particles of interest suspended in an aqueous solution. Light rays which strike the particles are scattered through angles which are inversely proportional to the particle size and a photodetector array measures the intensity of light at several predetermined angles and the measured intensities at different angles are processed by a computer using standard theoretical principles to determine the particle size distribution. Laser diffraction values may be obtained using a wet dispersion of the particles in distilled water.

Other methods that are commonly used in the art to measure Dᵥ10, Dᵥ50 and Dᵥ90 include disc centrifugation, scanning electron microscope (SEM), sedimentation field flow fractionation and photon correlation spectroscopy.

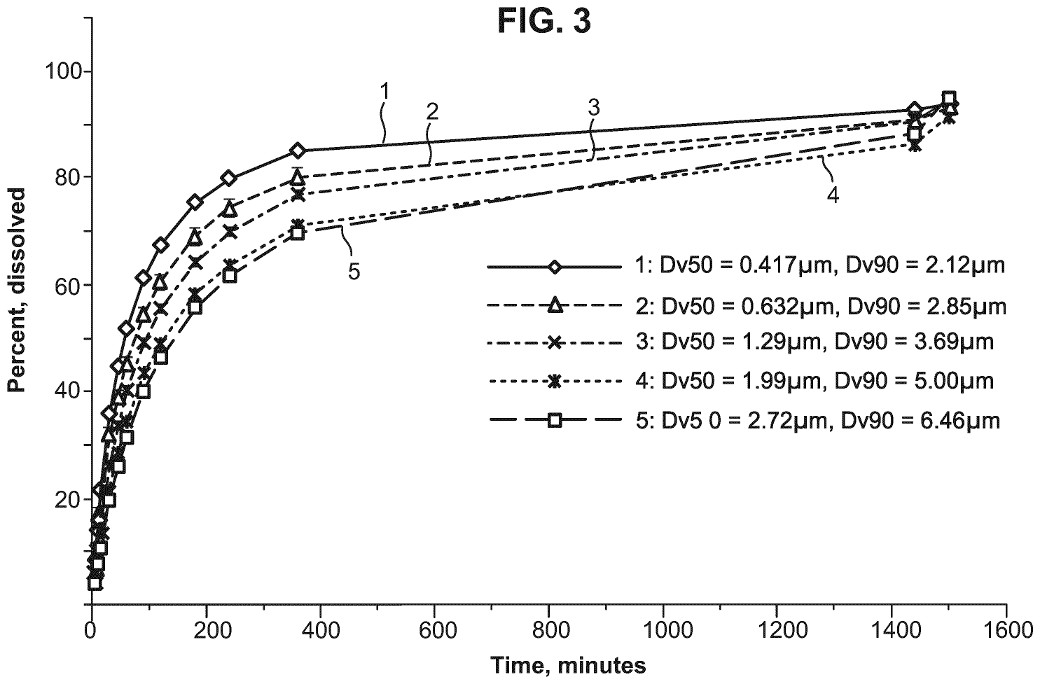

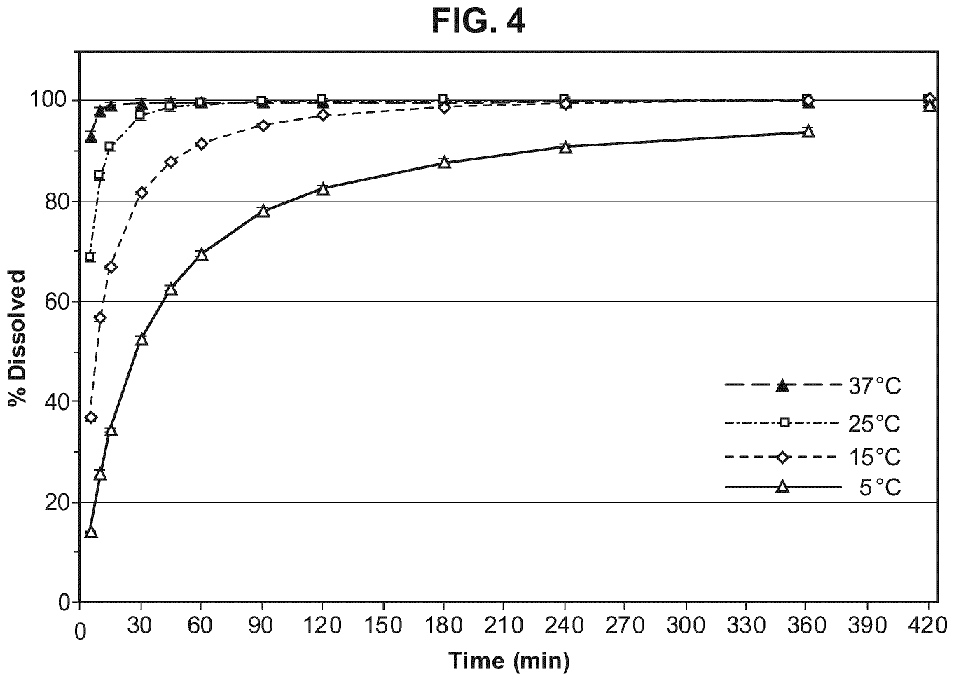

Samples with a larger particle size were found to have a slower rate of dissolution of the rilpivirine or a pharmaceutically acceptable salt thereof in the aqueous medium than samples with a lower particle size (see FIG. 3). Increasing the temperature of the aqueous medium increases the rate of dissolution (see FIG. 4). Accordingly, the temperature at which the aqueous medium is maintained may be further optimised based on the expected particle size distribution of the rilpivirine or a pharmaceutically acceptable salt thereof in the form of micro- or nanoparticles. The aqueous medium may be maintained at a higher temperature when testing samples of higher particle sizes in order to provide results within a reasonable timescale (e.g, wherein over 85% of the drug substance is dissolved after 6 hours). The aqueous medium may be maintained at a lower temperature when testing samples of lower particle sizes since this will still provide results within a reasonable timescale while the low temperature improves the discriminative properties of the test. For example, when testing samples according to the first preferred rilpivirine or pharmaceutically acceptable salt thereof particle size embodiment, the aqueous medium may be maintained at a temperature of 3-10° C., or 4-6° C., or 4.5-5.5° C. When testing samples according to the second preferred rilpivirine or pharmaceutically acceptable salt thereof particle size embodiment, the aqueous medium may be maintained at a temperature of 7-15° C. or 10-15° C.

In an embodiment, the rilpivirine or pharmaceutically acceptable salt thereof micro- or nanoparticles have one or more surface modifiers adsorbed to their surface.

The surface modifier may be selected from known organic and inorganic pharmaceutical excipients, including various polymers, low molecular weight oligomers, natural products and surfactants. Particular surface modifiers that may be used in the invention include nonionic and anionic surfactants. Representative examples of surface modifiers include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives; polyoxyethylene stearates, colloidal silicon dioxide, sodium dodecylsulfate, carboxymethylcellulose sodium, bile salts such as sodium taurocholate, sodium desoxytaurocholate, sodium desoxycholate; methylcellulose, hydroxyethylcellulose, hydroxypropylcellulose, hydroxypropylmethylcellulose, magnesium aluminate silicate, polyvinyl alcohol (PVA), poloxamers, such as Pluronic™ F68, F108 and F127 which are block copolymers of ethylene oxide and propylene oxide; tyloxapol; Vitamin E-TGPS (α-tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters (Tweens™ 20, 40, 60 and 80); sorbitan esters of fatty acids (Span™ 20, 40, 60 and 80 or Arlacel™ 20, 40, 60 and 80); polyethylene glycols (such as those sold under the tradename Carbowax™ 3550 and 934); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP). If desired, two or more surface modifiers can be used in combination.

In an embodiment, the surface modifier is selected from a poloxamer, α-tocopheryl polyethylene glycol succinate, polyoxyethylene sorbitan fatty acid ester, and salts of negatively charged phospholipids or the acid form thereof. In a preferred embodiment, the surface modifier is selected from Pluronic™ F108, Vitamin E TGPS (α-tocopheryl polyethylene glycol succinate, in particular α-tocopheryl polyethylene glycol 1000 succinate), polyoxyethylene sorbitan fatty acid esters such as Tween™ 80, and phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG.

In a preferred embodiment, the surface modifier is a poloxamer, in particular Pluronic™ F108. Pluronic™ F108 corresponds to poloxamer 338 and is the polyoxyethylene, polyoxypropylene block copolymer that conforms generally to the formula HO—[CH₂CH₂O]ₓ—[CH(CH₃)CH₂O]_{y}—[CH₂CH₂O]_{z}—H in which the average values of x, y and z are respectively 128, 54 and 128. Other commercial names of poloxamer 338 are Hodag Nonionic™ 1108-F and Synperonic™ PE/F108. In one embodiment, the surface modifier comprises a combination of a polyoxyethylene sorbitan fatty acid ester and a phosphatidyl glycerol salt (in particular egg phosphatidyl glycerol sodium).

In an embodiment, the relative amount (w/w) of rilpivirine or a pharmaceutically acceptable salt thereof to the surface modifier in the sample or in the batch is from about 1:2 to about 20:1, in particular from about 1:1 to about 10:1, e.g. from about 4:1 to about 6:1, preferably about 6:1.

In an embodiment, the micro- or nanoparticles of the invention comprise rilpivirine or a pharmaceutically acceptable salt thereof as defined herein and one or more surface modifiers as defined herein wherein the amount of rilpivirine or a pharmaceutically acceptable salt thereof is at least about 50% by weight of the micro- or nanoparticles, at least about 80% by weight of the micro- or nanoparticles, at least about 85% by weight of the micro- or nanoparticles, at least about 90% by weight of the micro- or nanoparticles, at least about 95% by weight of the micro- or nanoparticles, or at least about 99% by weight of the micro- or nanoparticles, in particular ranges between 80% and 90% by weight of the micro- or nanoparticles or ranges between 85% and 90% by weight of the micro- or nanoparticles.

The sample or batch of rilpivirine or a pharmaceutically acceptable salt thereof in the form of micro- or nanoparticles is preferably in the form of a suspension comprising a pharmaceutically acceptable aqueous carrier in which the micro- or nanoparticles are suspended. The pharmaceutically acceptable aqueous carrier comprises sterile water, e.g. water for injection, optionally in admixture with other pharmaceutically acceptable ingredients. The latter comprise any ingredients for use in injectable formulations. These ingredients may be selected from one or more of a suspending agent, a buffer, a pH adjusting agent, a preservative, an isotonizing agent, a surface modifier, a chelating agent and the like ingredients. In one embodiment, said ingredients are selected from one or more of a suspending agent, a buffer, a pH adjusting agent, and optionally, a preservative and an isotonizing agent. Particular ingredients may function as two or more of these agents simultaneously, e.g. behave like a preservative and a buffer, or behave like a buffer and an isotonizing agent. In an embodiment said ingredients are selected from one or more of a buffer, a pH adjusting agent, an isotonizing agent, a chelating agent and a surface modifier. In an embodiment said ingredients are selected from one or more of a buffer, a pH adjusting agent, an isotonizing agent, and a chelating agent.

In an embodiment, the suspension is formulated for administration by subcutaneous or intramuscular injection. In an embodiment, the suspension is formulated for administration by subcutaneous injection. In an embodiment, the suspension is formulated for administration by intramuscular injection.

In an embodiment, the suspension additionally comprises a buffering agent and/or a pH adjusting agent. Suitable buffering agents and pH adjusting agents should be used in amount sufficient to render the suspension in the pH range of 6 to pH 8.5, preferably in the pH range of 7 to 7.5. Particular buffers are the salts of weak acids. Buffering and pH adjusting agents that can be added may be selected from tartaric acid, maleic acid, glycine, sodium lactate/lactic acid, ascorbic acid, sodium citrates/citric acid, sodium acetate/acetic acid, sodium bicarbonate/carbonic acid, sodium succinate/succinic acid, sodium benzoate/benzoic acid, sodium phosphates, tris(hydroxymethyl) aminomethane, sodium bicarbonate/sodium carbonate, ammonium hydroxide, benzene sulfonic acid, benzoate sodium/acid, diethanolamine, glucono delta lactone, hydrochloric acid, hydrogen bromide, lysine, methanesulfonic acid, monoethanolamine, sodium hydroxide, tromethamine, gluconic, glyceric, gluratic, glutamic, ethylene diamine tetraacetic (EDTA), triethanolamine, including mixtures thereof. In an embodiment, the buffer is a sodium phosphate buffer, e.g. sodium dihydrogen phosphate monohydrate. In an embodiment the pH adjusting agent is sodium hydroxide.

In an embodiment, the suspension additionally comprises a preservative. Preservatives comprise antimicrobials and anti-oxidants which can be selected from the group consisting of benzoic acid, benzyl alcohol, butylated hydroxyanisole (BHA), butylated hydroxytoluene (BHT), chlorbutol, a gallate, a hydroxybenzoate, EDTA, phenol, chlorocresol, metacresol, benzethonium chloride, myristyl-γ-piccolinium chloride, phenylmercuric acetate and thimerosal. Radical scavengers include BHA, BHT, Vitamin E and ascorbyl palmitate, and mixtures thereof. Oxygen scavengers include sodium ascorbate, sodium sulfite, L-cysteine, acetylcysteine, methionine, thioglycerol, acetone sodium bisulfite, isoacorbic acid, hydroxypropyl cyclodextrin. Chelating agents include sodium citrate, sodium EDTA, citric acid and malic acid. In an embodiment, the chelating agent is citric acid, e.g. citric acid monohydrate.

In an embodiment, the suspension additionally comprises an isotonizing agent. An isotonizing agent or isotonifier may be present to ensure isotonicity of the pharmaceutical compositions of the present invention, and includes sugars such as glucose, dextrose, sucrose, fructose, trehalose, lactose; polyhydric sugar alcohols, preferably trihydric or higher sugar alcohols, such as glycerin, erythritol, arabitol, xylitol, sorbitol and mannitol. Alternatively, sodium chloride, sodium sulfate, or other appropriate inorganic salts may be used to render the solutions isotonic. These isotonifiers can be used alone or in combination. The suspensions conveniently comprise from 0 to 10% (w/v), in particular 0 to 6% (w/v) of isotonizing agent. Of interest are nonionic isotonifiers, e.g. glucose, mannitol, as electrolytes may affect colloidal stability.

In an embodiment, the batch contains multiple doses of rilpivirine or a pharmaceutically acceptable salt thereof formulated to be suitable for administration by intramuscular or subcutaneous injection, optionally for the long-term treatment of HIV infection in a subject infected with HIV or for the long-term prevention of HIV infection in a subject at risk of being infected by HIV.

In an embodiment, the batch contains multiple doses formulated such that each dose comprises up to about 150 mL of the suspension described herein, i.e. the volume of the suspension comprising the rilpivirine or a pharmaceutically acceptable salt thereof in the form of micro- or nanoparticles may have a volume of up to 150 mL. In an embodiment, each dose comprises from about 2 mL to about 100 mL of the suspension. In another embodiment, each dose comprises from about 3 mL to about 75 mL of the suspension. In another embodiment, each dose comprises from about 4 mL to about 50 mL of the suspension. In another embodiment, each dose comprises from about 5 mL to about 25 mL of the suspension. In another embodiment, each dose comprises from about 6 mL to about 20 mL of the suspension. In another embodiment, each dose comprises from about 6 mL to about 18 mL of the suspension. In another embodiment, each dose comprises from about 6 mL to about 15 mL of the suspension. In another embodiment, each dose comprises from about 6 mL to about 12 mL of the suspension. In another embodiment, each dose comprises from about 9 mL to about 18 mL of the suspension. In another embodiment, each dose comprises from about 9 mL to about 15 mL of the suspension. In another embodiment, each dose comprises from about 9 mL to about 12 mL of the suspension. In another embodiment, each dose comprises about 6 mL of the suspension. In another embodiment, each dose comprises about 9 mL of the suspension. In another embodiment, each dose comprises about 12 mL of the suspension. In another embodiment, each dose comprises about 15 mL of the suspension. In another embodiment, each dose comprises about 18 mL of the suspension. In an embodiment, the rilpivirine suspension contains 300 mg rilpivirine or pharmaceutically acceptable salt thereof/mL. In an embodiment, the rilpivirine suspension contains 300 mg rilpivirine or pharmaceutically acceptable salt thereof/mL and the dose is 2 mL. In an embodiment, the rilpivirine suspension contains 300 mg rilpivirine or pharmaceutically acceptable salt thereof/mL and the dose is 3 mL.

In an embodiment, when the rilpivirine or a pharmaceutically acceptable salt thereof in the form of micro- or nanoparticles is for the treatment of HIV infection, the batch contains multiple doses formulated such that the dose to be administered may be calculated on a basis of about 300 mg to about 1200 mg/month, or about 450 mg to about 1200 mg/month, or about 450 mg to about 900 mg/month, or about 600 mg to about 900 mg/month, or about 450 mg to about 750 mg/month, or 450 mg/month, or 600 mg/month, or 750 mg/month, or 900 mg/month. Doses for other dosing regimens can readily be calculated by multiplying the monthly dose with the number of months between each administration. For example, in case of a dose of 450 mg/month, and in case of a time interval of 6 months between each administration, the dose to be administered in each administration is 2700 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment, for the treatment of HIV infection, the batch contains multiple doses formulated such that the dose to be administered may be calculated on a basis of about 300 mg to about 1200 mg/4 weeks (28 days), or about 450 mg to about 1200 mg/4 weeks (28 days), or about 450 mg to about 900 mg/4 weeks (28 days), or about 600 mg to about 900 mg/4 weeks (28 days), or about 450 mg to about 750 mg/4 weeks (28 days) or 450 mg/4 weeks (28 days), or 600 mg/4 weeks (28 days), or 750 mg/4 weeks (28 days) or 900 mg/4 weeks (28 days). Doses for other dosing regimens can readily be calculated by multiplying the week or day dose with the number of weeks between each administration. For example, in case of a dose of 450 mg/4 weeks (28 days), and in case of a time interval of 24 weeks between each administration, the dose to be administered in each administration is 2700 mg. Or for example, in case of a dose of 750 mg/4 weeks (28 days), and in case of a time interval of 24 weeks between each administration, the dose to be administered in each administration is 4500 mg. The indicated “mg” corresponds to mg of rilpivirine. Thus, by way of example, 1 mg of rilpivirine corresponds to 1.1 mg of rilpivirine hydrochloride.

In an embodiment, for the treatment of HIV infection, the batch contains multiple doses formulated such that each dose of rilpivirine or a pharmaceutically acceptable salt thereof may comprise at least about 600 mg, such as from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg or from about 2700 mg to about 4500 mg of the rilpivirine or pharmaceutically acceptable salt thereof.

Thus, the amount of the rilpivirine or pharmaceutically acceptable salt thereof in the doses in the batch may be at least about 600 mg, such as from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg or from about 2700 mg to about 4500 mg. The indicated “mg” corresponds to mg of rilpivirine. Thus, by way of example, 1 mg of rilpivirine corresponds to 1.1 mg of rilpivirine hydrochloride. In an embodiment, the amount of rilpivirine or a pharmaceutically acceptable salt thereof in the dose is 600 mg. In an embodiment, the amount of rilpivirine or a pharmaceutically acceptable salt thereof in the dose is 900 mg.

In the instance of prevention of HIV infection, each administration of rilpivirine or pharmaceutically acceptable salt thereof may comprise the same dosing as for therapeutic applications as described above.

In an embodiment, the doses in the batch are formulated such that, in use, preferably for treatment of HIV infection, in particular HIV-1 infection, the blood plasma concentration of rilpivirine in the subject is kept at a level above about 12 ng/ml, preferably ranging from about 12 ng/ml to about 100 ng/ml, more preferably about 12 ng/ml to about 50 ng/ml for at least one month, or two months or three months after administration, or at least 6 months after administration, or at least 9 months after administration, or at least 1 year after administration, or at least 2 years after each administration. In an embodiment, the doses in the batch are formulated such that, in use, the blood plasma concentration of rilpivirine in the subject is kept at a level of from 12 ng/ml to 100 ng/ml for one month. In an embodiment, the doses in the batch are formulated such that, in use, the blood plasma concentration of rilpivirine in the subject is kept at a level of from 12 ng/ml to 100 ng/ml for two months. In an embodiment, the doses in the batch are formulated such that, in use, the blood plasma concentration of rilpivirine in the subject is kept at a level of from 12 ng/ml to 100 ng/ml for at least 6 months.

In an embodiment, the batch contains multiple doses formulated for administration, preferably by subcutaneous or intramuscular injection, intermittently at a time interval in the range of 1 week to 2 years, or 2 weeks to 1 year, or 1 month to 6 months, or about 1 month, or about 2 months, or about 3 months, or about 4 months, or about 5 months, or about 6 months.

In a particular embodiment, the sample or batch of rilpivirine or a pharmaceutically acceptable salt thereof in the form of micro- or nanoparticles is formulated as a suspension comprising one or more of, optionally all of, the following components:
rilpivirine or a pharmaceutically acceptable salt thereof, in particular rilpivirine;
a surface modifier as defined herein, in particular poloxamer 338;
an isotonizing agent, in particular glucose monohydrate;
a buffer, in particular sodium dihydrogen phosphate;
a chelating agent, in particular citric acid monohydrate;
a pH adjusting agent, in particular sodium hydroxide; and
water, in particular water for injection.

In another particular embodiment, the sample or batch of rilpivirine or a pharmaceutically acceptable salt thereof in the form of micro- or nanoparticles is formulated as a suspension comprising one or more of, optionally all of, the following components:
rilpivirine or a pharmaceutically acceptable salt thereof, in particular rilpivirine;
poloxamer 338;
glucose monohydrate;
sodium dihydrogen phosphate;
citric acid monohydrate;
sodium hydroxide; and
water, in particular water for injection.

In one embodiment, the sample or batch of rilpivirine or a pharmaceutically acceptable salt thereof in the form of micro- or nanoparticles is formulated as an aqueous suspension comprising by weight, based on the total volume of the suspension:
(a) from 3% to 50% (w/v), or from 10% to 40% (w/v), or from 10% to 30% (w/v), of rilpivirine or a pharmaceutically acceptable salt thereof; in particular rilpivirine;
(b) from 0.5% to 10% (w/v), or from 0.5% to 5% (w/v), or from 0.5% to 2% (w/v) of a surface modifier; in particular poloxamer 338;
(c) from 0% to 10% (w/v), or from 0% to 5% (w/v), or from 0% to 2% (w/v), or from 0% to 1% (w/v) of one or more buffering agents; in particular sodium dihydrogen phosphate;
(d) from 0% to 10% (w/v), or from 0% to 6% (w/v), or from 0% to 5% (w/v), or from 0% to 3% (w/v), or from 0% to 2% (w/v) of an isotonizing agent; in particular glucose monohydrate;
(e) from 0% to 2% (w/v), or from 0% to 1% (w/v), or from 0% to 0.5% (w/v), or from 0% to 0.1% (w/v) of a pH adjusting agent; in particular sodium hydroxide;
(f) from 0% to 2% (w/v), or from 0% to 1% (w/v), or from 0% to 0.5% (w/v), or from 0% to 0.1% (w/v) of a chelating agent; in particular citric acid monohydrate;
(g) from 0% to 2% (w/v) preservatives; and
(h) water for injection q.s. ad 100%.

In one embodiment, the aqueous suspensions may comprise by weight, based on the total volume of the suspension:
(a) from 3% to 50% (w/v), or from 10% to 40% (w/v), or from 10% to 30% (w/v), of rilpivirine or a pharmaceutically acceptable salt thereof; in particular rilpivirine;
(b) from 0.5% to 10% (w/v), or from 0.5% to 5% (w/v), or from 0.5% to 2% (w/v) of a surface modifier; in particular poloxamer 338;
(c) from 0% to 10% (w/v), or from 0% to 5% (w/v), or from 0% to 2% (w/v), or from 0% to 1% (w/v) of one or more buffering agents; in particular sodium dihydrogen phosphate;
(d) from 0% to 10% (w/v), or from 0% to 6% (w/v), or from 0% to 5% (w/v), or from 0% to 3% (w/v), or from 0% to 2% (w/v) of an isotonizing agent; in particular glucose monohydrate;
(e) from 0% to 2% (w/v), or from 0% to 1% (w/v), or from 0% to 0.5% (w/v), or from 0% to 0.1% (w/v) of a pH adjusting agent; in particular sodium hydroxide;
(f) from 0% to 2% (w/v), or from 0% to 1% (w/v), or from 0% to 0.5% (w/v), or from 0% to 0.1% (w/v) of a chelating agent; in particular citric acid monohydrate; and
(g) water for injection q.s. ad 100%.

In a particular embodiment, the sample or batch of rilpivirine or pharmaceutically acceptable salt thereof is formulated as a suspension of micro- or nanoparticles wherein the suspension comprises the following components in the following amounts:
(a) Rilpivirine (300 mg);
(b) Poloxamer 338 (50 mg); and
(c) Water for injection (ad 1 ml).

Alternatively, these components may be used in different amounts but with the same weight ratio between components and the total volume (made up by water for injection) scaled by the same value.

In a particular embodiment, the sample or batch of rilpivirine or pharmaceutically acceptable salt thereof is formulated (and administered) as a suspension of micro- or nanoparticles wherein the suspension comprises the following components in the following amounts:
a. Rilpivirine (300 mg);
b. Poloxamer 338 (50 mg);
c. Glucose monohydrate (19.25 mg);
d. Sodium dihydrogen phosphate (2.00 mg);
e. Citric acid monohydrate (1.00 mg);
f. Sodium Hydroxide (0.866 mg); and
g. Water for injection (ad 1 ml).

Alternatively, these components may be used in different amounts but with the same weight ratio between components and the total volume (made up by water for injection) scaled by the same value.

In an embodiment, the suspension of rilpivirine or a pharmaceutically acceptable salt thereof as described herein is suitable for administration by a manual injection process.

As used herein the term “treatment of HIV infection” relates to the treatment of a subject infected with HIV, in particular HIV-1. The term “treatment of HIV infection” also relates to the treatment of diseases associated with HIV infection, for example AIDS, or other conditions associated with HIV infection including thrombocytopenia, Kaposi's sarcoma and infection of the central nervous system characterized by progressive demyelination, resulting in dementia and symptoms such as, progressive dysarthria, ataxia and disorientation, and further conditions where HIV infection has also been associated with, such as peripheral neuropathy, progressive generalized lymphadenopathy (PGL), and AIDS-related complex (ARC).

As used herein the term “prevention of HIV infection” relates to the prevention or avoidance of a subject (who is not infected with HIV) becoming infected with HIV, in particular HIV-1. The source of infection can be various, a material containing HIV, in particular a body fluid that contains HIV such as blood or semen, or another subject who is infected with HIV. Prevention of HIV infection relates to the prevention of the transmission of the virus from the material containing HIV or from the HIV infected individual to an uninfected person, or relates to the prevention of the virus from entering the body of an uninfected person. Transmission of the HIV virus can be by any known cause of HIV transfer such as by sexual transmission or by contact with blood of an infected subject, e.g. medical staff providing care to infected subjects. Transfer of HIV can also occur by contact with HIV infected blood, e.g. when handling blood samples or with blood transfusion. It can also be by contact with infected cells, e.g. when carrying out laboratory experiments with HIV infected cells.

The term “treatment of HIV infection” refers to a treatment by which the viral load of HIV (represented as the number of copies of viral RNA in a specified volume of serum) is reduced. The more effective the treatment, the lower the viral load. Preferably the viral load should be reduced to as low levels as possible, e.g. below about 200 copies/mL, in particular below about 100 copies/mL, more in particular below 50 copies/mL, if possible below the detection limit of the virus. Reductions of viral load of one, two or even three orders of magnitude (e.g. a reduction in the order of about 10 to about 102, or more, such as about 103) are an indication of the effectiveness of the treatment. Another parameter to measure effectiveness of HIV treatment is the CD4 count, which in normal adults ranges from 500 to 1500 cells per μL. Lowered CD4 counts are an indication of HIV infection and once below about 200 cells per μL, AIDS may develop. An increase of CD4 count, e.g. with about 50, 100, 200 or more cells per μL, is also an indication of the effectiveness of anti-HIV treatment. The CD4 count in particular should be increased to a level above about 200 cells per μL, or above about 350 cells per μL. Viral load or CD4 count, or both, can be used to diagnose the degree of HIV infection. Another parameter to measure effectiveness of HIV treatment is keeping the HIV-infected subject virologically suppressed (HIV-1 RNA<50 copies/mL) when on the treatment according to the present invention.

The term “treatment of HIV infection” and similar terms refer to that treatment that lowers the viral load, increases CD4 count, or both, or keeps the HIV-infected subject virologically suppressed, as described above. The term “prevention of HIV infection” and similar terms refer to that situation where there is a decrease in the relative number of newly infected subjects in a population in contact with a source of HIV infection such as a material containing HIV, or a HIV infected subject. Effective prevention can be measured, for example, by measuring in a mixed population of HIV infected and non-infected individuals, if there is a decrease of the relative number of newly infected individuals, when comparing non-infected individuals treated with a pharmaceutical composition of the invention, and non-treated non-infected individuals. This decrease can be measured by statistical analysis of the numbers of infected and non-infected individuals in a given population over time.

### General Definitions

The term “comprising” encompasses “including” as well as “consisting”, e.g. a composition “comprising” X may consist exclusively of X or may include something additional, e.g. X+Y. The term “comprising” used herein also encompasses “consisting essentially of”, e.g. a composition “comprising” X may consist of X and any other components that do not materially affect the essential characteristics of the composition.

The term “about” in relation to a numerical value Y is optional and means, for example, Y ±10%.

When a time interval is expressed as a specified number of months, it runs from a given numbered day of a given month to the same numbered day of the month that falls the specified number of months later. Where the same numbered day does not exist in the month that falls the specified number of months later, the time interval runs into the following month for the same number of days it would have run if the same numbered day would exist in the month that falls the specified number of months later.

When a time interval is expressed as a number of years, it runs from a given date of a given year to the same date in the year that falls the specified number of years later. Where the same date does not exist in the year that falls the specified number of years later, the time interval runs for the same number of days it would have run if the same numbered day would exist in the month that falls the specified number of months later. In other words, if the time interval starts on 29th February of a given year but ends in a year where there is no 29th February, the time period ends instead on 1st March in that year. The term “about” in relation to such a definition means that the time interval may end on a date that is ±10% of the time interval.

In an embodiment, the time interval may start up to 7 days before or after the start of the time interval and end up to 7 days before or after the end of the time interval.

All references cited herein are incorporated by reference in their entirety.

The invention will now be described with reference to the following examples. For the avoidance of doubt, these examples do not limit the scope of the invention. Modifications may be made whilst remaining within the scope and spirit of the invention.

### EXAMPLES

### Example 1—Discriminating Between Different Particle Sizes

The ability of the dissolution test to discriminate between different particle sizes of rilpivirine was explored. Suspensions of 300 mg/mL rilpivirine with the following excipients were prepared:
Poloxamer 338 (50 mg/ml)
Glucose monohydrate (19.25 mg/ml)
Sodium dihydrogen phosphate monohydrate (2.00 mg/ml)
Citric acid monohydrate (1.00 mg/ml)
Sodium hydroxide (0.866 mg/ml)
Water for injection (q.s ad 1 mL)

The particle size distribution of the rilpivirine was varied by controlling the milling parameters used when preparing the suspensions, and determined using laser diffraction:

TABLE 1
Particle sizes

| Suspension | Dᵥ10 (nm) | Dᵥ50 (nm) | Dᵥ90 (nm) |
|---|---|---|---|
| (a) | 198 | 3060 | 19483 |
| (b) | 132 | 1403 | 8207 |
| (c) | 81 | 286 | 2747 |
| (d) | 79 | 231 | 1765 |
| (e) | 79 | 223 | 1333 |
| (f) | 78 | 204 | 971 |
| (g) | 78 | 196 | 659 |
| (h) | 76 | 184 | 540 |
| (i) | 75 | 174 | 434 |

The dissolution of the suspensions was tested using a paddle apparatus (USP type 2, Ph. Eur, JP) with a rotation speed of 50 rpm in 900 mL 6.0% w/v polysorbate 20 in 0.05 M sodium phosphate buffer pH 7.4 at 5° C. The sample amount corresponds to 18 mg rilpivirine.

After 360 minutes the temperature was increased to 37° C. and maintained for 60 minutes to simulate an infinity time point. The samples taken after the temperature infinity time points are labelled as 420 minutes.

The quantity of dissolved drug substance was determined by a gradient ultra-high performance liquid chromatographic (UHPLC) method with UV detection at 280 nm.

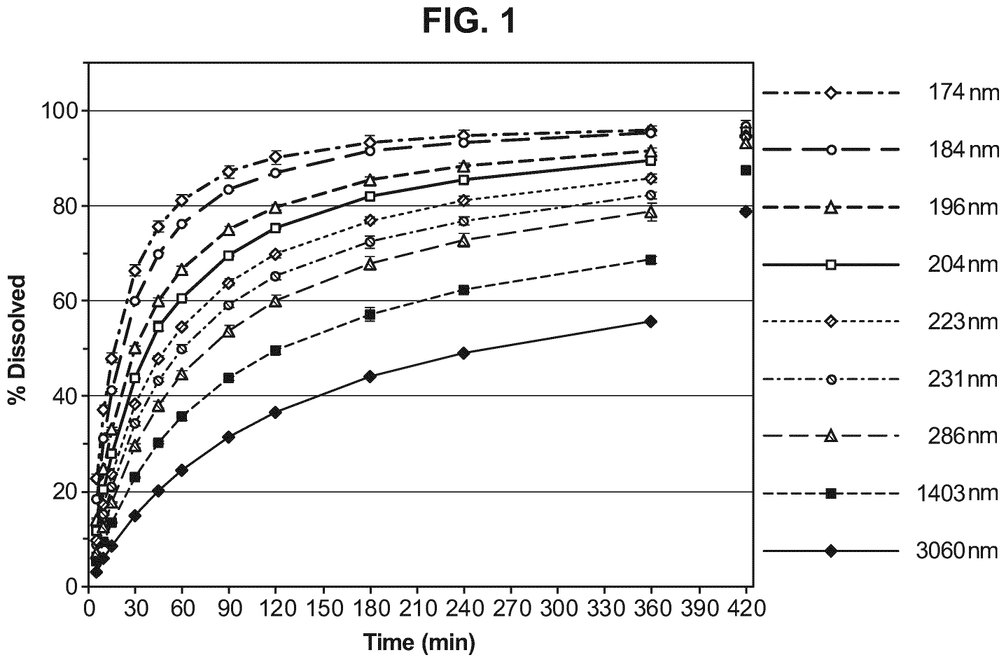

The results are shown in FIG. 1. It was found that the dissolution method was discriminating for the particle size distribution of the drug substance particles, which determines the behaviour of the drug substance in vivo. Surprisingly, the method was discriminating even between samples at small particle sizes, e.g. with suspension (h) with Dᵥ50=184 nm readily discriminated from suspension (i) with Dᵥ50=174 nm. Accordingly, the dissolution method provides a convenient method of quality control of batches of rilpivirine or a pharmaceutically acceptable salt thereof in the form of micro- or nanoparticles.

### Example 2—Discriminating Between Different Particle Sizes

This example compares the dissolution profile of three rilpivirine suspensions, each having a different particle size.

### Suspension 1

A 3.380 mL fill of 300 mg/mL suspension of rilpivirine (Dᵥ50=˜200 nm) was prepared in 4R glass vials with the following excipients:
Poloxamer 338 (50 mg/ml)
Glucose monohydrate (19.25 mg/ml)
Sodium dihydrogen phosphate monohydrate (2.00 mg/ml)
Citric acid monohydrate (1.00 mg/ml)
Sodium hydroxide (0.866 mg/ml)
Water for injection (q.s ad 3 mL)

The suspension was prepared as follows:

A buffer solution was prepared by dissolving citric acid monohydrate, sodium dihydrogen phosphate monohydrate, sodium hydroxide and, glucose monohydrate in water for injection in a stainless steel vessel. Poloxamer 338 was added to the buffer solution and mixed until dissolved. A first fraction of the poloxamer 338 buffer solution was passed sequentially through a pre-filter and 2 serially-connected sterile filters into a sterilized stainless steel vessel. The sterile drug substance (micronized irradiated) was aseptically dispersed, via a charging isolator, into the sterile solution. The remaining fraction of poloxamer 338 buffer solution was passed sequentially through a pre-filter and 2 serially-connected sterile filters into the milling vessel to make up the suspension concentrate. During and after addition of the drug substance, the suspension concentrate was mixed to wet and disperse the drug substance.

### Milling of the Suspension Concentrate

The suspension concentrate in the milling vessel was aseptically milled by circulating through a sterilized stainless-steel milling chamber, using sterilized zirconia beads as grinding media. During the milling process, the suspension circulated between the milling chamber and the milling vessel by means of a peristaltic pump until the target particle size was achieved.

### Dilution of the Suspension Concentrate to Final Concentration

The suspension concentrate in the holding vessel was diluted with water for injection, which is sterile filtered through a pre-filter and 2 serially connected sterile filters into this vessel via the milling chamber and the 70 μm stainless steel filter. After final dilution, the vessel headspace is blanketed with nitrogen and the suspension was mixed until homogeneous.

### Holding and Filling of the Final Suspension

While mixing, the suspension was aseptically transferred from the holding vessel to the time/pressure (t/p) dosing vessel, from which the suspension was filled into vials which were flushed with nitrogen, stoppered and capped with an aluminium seal with a flip-off button.

### Suspensions 2 and 3

Two further suspensions, having the same composition but different particle sizes, were prepared by compounding and milling (suspensions 2 and 3) as described below.
1. 586.62 g water for injection was added to a 2 L glass beaker containing a magnetic stir bar.
2. The correct amount of citric acid monohydrate, sodium dihydrogen phosphate monohydrate, sodium hydroxide was added and stirred until dissolved.
3. The correct amount of poloxamer 338 and glucose monohydrate was added and stirred until dissolved.
4. The diluent was filtered through a 0.22 μm filter, the beaker was rinsed with the remaining 100 mL water for injection and filtered.
5. Rilpivirine microfine was added and stirred until a homogenous suspension was obtained.
6. 500 mL of the suspension was transferred in sterilized beaker and placed in a double walled cooled glass beaker with magnetic stir bar.
7. Start milling on Netzsch Labstar, mill until target particle size distribution is reached. For suspension 2, milling time was about 180 minutes. For suspension 3, milling time was about 35 minutes.
8. The particle size distribution was measured during milling.
9. Each suspension was diluted to 300 mg/mL.

### Particle Size Distribution Measurement

The volume-based particle size distribution of the rilpivirine suspensions was determined by means of wet dispersion laser diffraction, using a Malvern Mastersizer 3000 laser diffraction (Malvern Instruments) and Hydro MV wet dispersion module.

The particle size of the three rilpivirine suspensions were as defined in Table 2.

TABLE 2
Particle sizes

| Suspension | Dᵥ50 (μm) | Dᵥ90 (μm) |
|---|---|---|
| 1 | 0.29 | 0.69 |
| 2 | 0.39 | 1.91 |
| 3 | 2.46 | 5.55 |

### In Vitro Dissolution Measurement

The dissolution of the three rilpivirine suspensions in water was performed using Paddle Apparatus (USP type 2, Ph.Eur., JP.) at 50 rpm in 900 mL of 6.0% w/v Polysorbate 20 in 0.05 M Sodium Phosphate buffer pH 7.4, at 5.0±0.5° C. An amount of 64.98 mg (=0.06 mL×1.083 g/mL (the theoretical density of the suspension))±5% of homogeneous suspension of rilpivirine (corresponding to 18±0.9 mg of rilpivirine) was added.

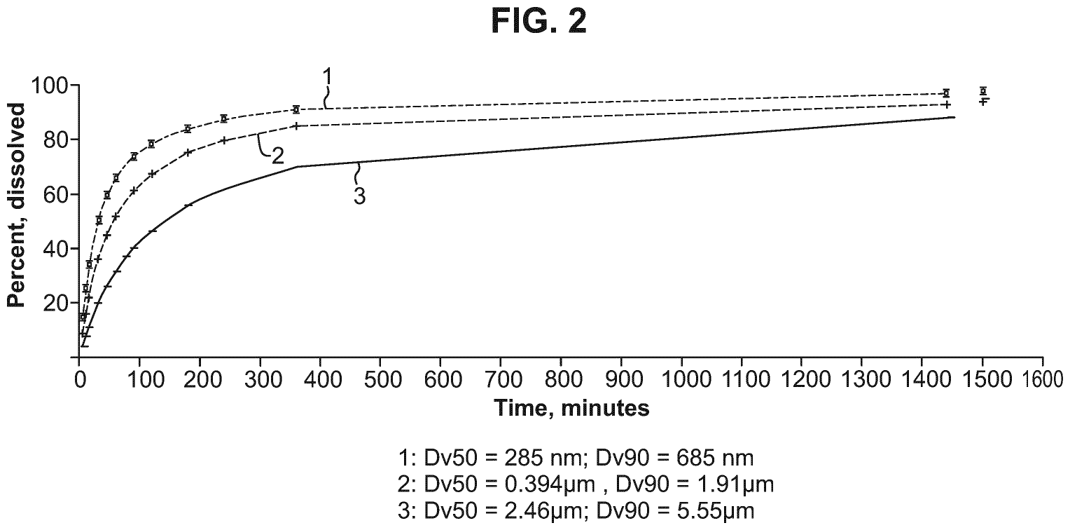

The determination of the quantity of rilpivirine present in the dissolution samples is based upon a gradient ultra-high performance liquid chromatographic (UHPLC) method with UV detection at 280 nm. Results are shown in FIG. 2, which demonstrates the ability of the dissolution test to discriminate between Suspensions 1, 2, and 3. The dissolution test shows that rilpivirine in the form of micro- or nanoparticles having larger particle sizes as shown in Table 2 surprisingly lowered, i.e. flattened, the dissolution profile of rilpivirine.

### Example 3—Discriminating Between Different Particle Sizes

This example compares the dissolution profile of five rilpivirine suspensions, each having a different particle size.

### Preparation of Rilpivirine Suspensions and Measurement of Particle Size

Five suspensions of rilpivirine were prepared according to a method corresponding to the method described for suspensions 2 and 3 in Example 2. The volume-based particle size distribution of the rilpivirine micro- or nanoparticles in suspension was determined according to a method corresponding to the method that is specified in Example 2.

TABLE 3
Particle sizes

| Suspension | Dᵥ50 (μm) | Dᵥ90 (μm) |
|---|---|---|
| 1 | 0.42 | 2.12 |
| 2 | 0.63 | 2.85 |
| 3 | 1.29 | 3.69 |
| 4 | 1.99 | 5.00 |
| 5 | 2.72 | 6.46 |

### In Vitro Dissolution Measurement

The dissolution of the five rilpivirine suspensions in water was performed according to the method that is specified in Example 2. Results are shown in FIG. 3, which demonstrate that the dissolution test can discriminate between different particle sizes of rilpivirine. As the particle size of rilpivirine in the form of micro- or nanoparticles is increased the dissolution profile of rilpivirine is lowered, i.e. flattened.

### Example 4—Dissolution Temperature

The dissolution of a suspension of nanoparticulate rilpivirine having a Dᵥ50 of ˜200 nm was tested using the method of Example 1 but with different temperatures of the dissolution medium (37° C., 25° C., 15° C., and 5° C.). The dissolution profiles are shown in FIG. 4.

The use of dissolution medium temperatures below the physiological temperature of 37° C. was found to be crucial to the ability of the dissolution test to discriminate between different particle sizes of rilpivirine. At 37° C. the rilpivirine is fully dissolved in around 10 minutes. However, the use of lower temperatures slowed the release of rilpivirine to such an extent that the discriminative power of the method was significantly increased. At 5° C. the drug substance is less than 30% dissolved at 5 minutes, thereby allowing the detection of potential initial increased (burst) release. Over 85% of the drug substance is dissolved after 6 hours, thereby affording a dissolution profile that is sufficiently varied to allow discrimination between different particle sizes of rilpivirine or a pharmaceutically acceptable salt thereof in the form of micro- or nanoparticles, over a practically convenient timescale.

### Example 5—Surfactant Concentration

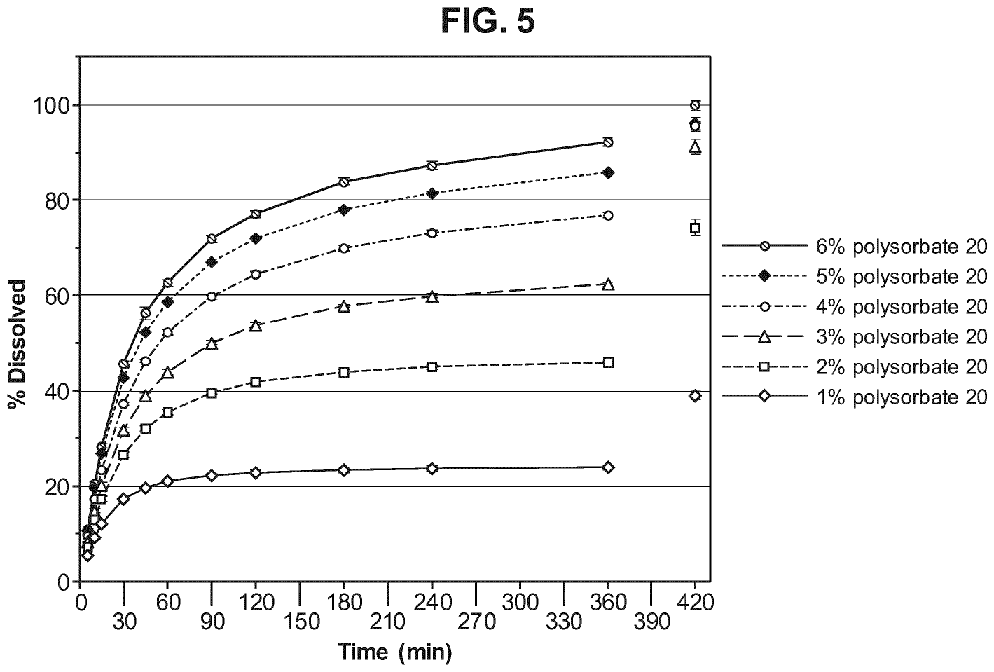

The dissolution of a suspension of nanoparticulate rilpivirine having a Dᵥ50 of 218 nm was tested using the method of Example 1 but with different concentrations of the surfactant (1-6% w/v polysorbate 20). The dissolution profiles are shown in FIG. 5.

The use of higher concentrations of polysorbate 20 resulted in dissolution profiles that are further optimised. For example, with the addition of 6% polysorbate 20 the initial release of rilpivirine is still well below 20% dissolved while after 360 minutes more than 85% dissolved can be reached. Consequently, this enables a single method to better detect a potential burst release, characterize the release profile, and detect final release above 50%, or 60%, or 70%, or 80%, or 90% dissolved, preferably 100% dissolved. The performance of each method can also be defined by calculating the difference between the lowest and highest % dissolved in the dissolution profile, i.e. the delta % dissolved. The delta % dissolved of the 6% polysorbate 20 method is approximately 80%. The higher the % dissolved, the higher the ability of the method to discriminate between different particle sizes of rilpivirine. Likewise, one could optimize the method by controlling the surfactant concentrations to increase the delta % dissolved.

### Example 6—Sink Conditions

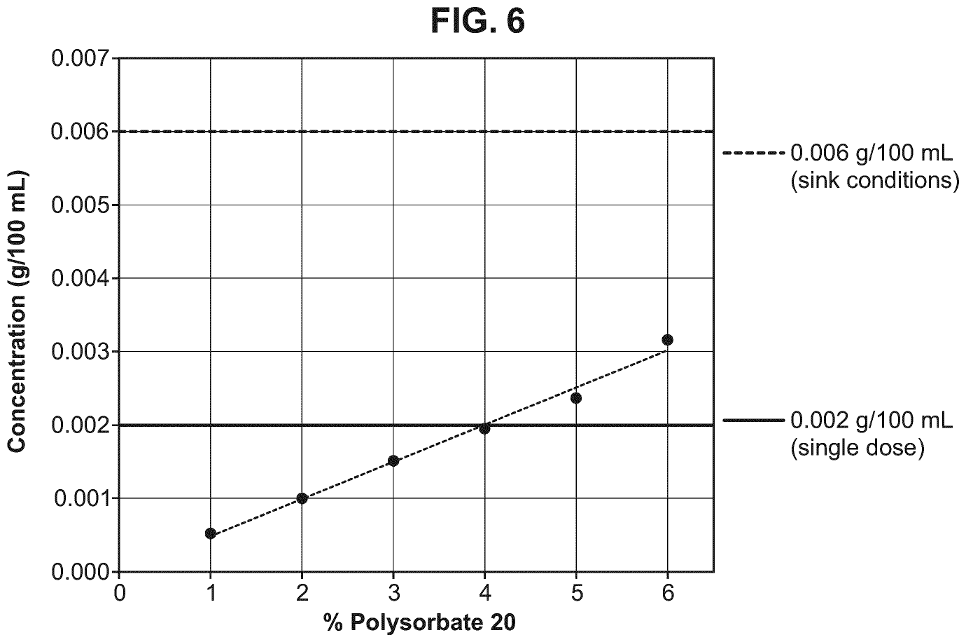

The equilibrium solubility of rilpivirine in 0.05 M phosphate buffer at pH 7.4 as a function of the concentration of polysorbate 20 was determined at 5° C. and is presented in FIG. 6. Reference lines show the concentration equivalent to a single dose of 18 mg of rilpivirine dissolved in the medium (0.002 g/100 mL, lower horizontal line), and sink conditions (defined as ≥3× the single dose, i.e. ≥0.006 g/100 mL, upper horizontal line). Although not at sink conditions, the dissolution test was found to discriminate between different particle sizes of rilpivirine, as shown by the examples herein.

### Example 7—Discriminating Between Storage Conditions

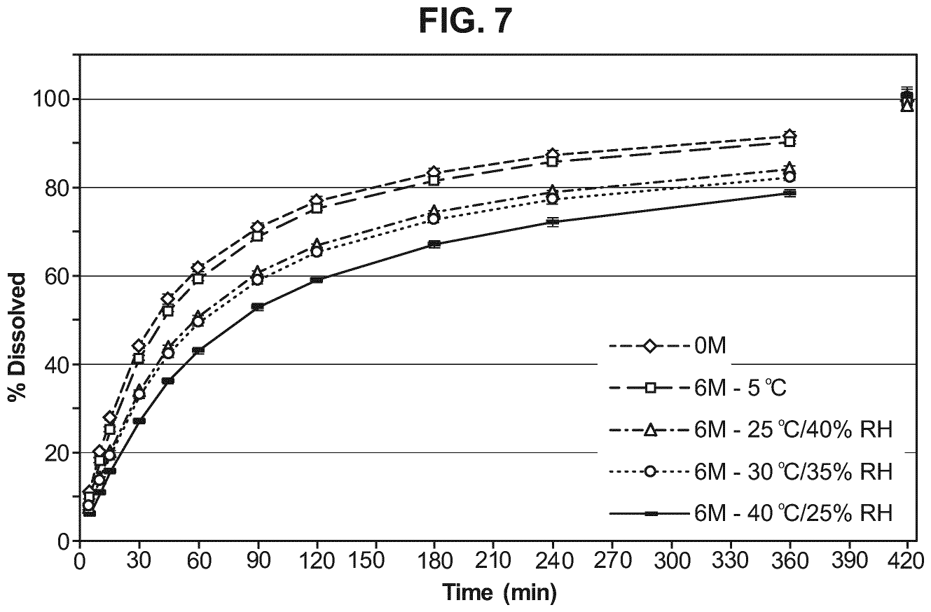

The dissolution of a suspension of nanoparticulate rilpivirine having a Dᵥ50 of 192 nm stored under different conditions was tested using the method of Example 1. The storage conditions were: 6 months at 5° C., and 6 months under accelerated stress conditions of 25° C./40% RH, 30° C./35% RH, and 40° C./25% RH. The dissolution profiles are shown in FIG. 7. It can be concluded that the dissolution method is able to detect changes to the drug product after exposure to stressed temperature and humidity conditions, since it was able to discriminate between samples stored in the different conditions tested.

1. A method of testing a sample of rilpivirine or a pharmaceutically acceptable salt thereof, wherein the sample comprises rilpivirine or a pharmaceutically acceptable salt thereof in the form of micro- or nanoparticles, the method comprising:
dispersing the sample into an aqueous medium, wherein the aqueous medium:
comprises a surfactant, and
is maintained at a temperature of 2-15° C.; and
measuring the dissolution of the rilpivirine or a pharmaceutically acceptable salt thereof in the aqueous medium.

2. The method of claim 1, wherein the aqueous medium is maintained at a temperature of 3-10° C., or 4-6° C., or 4.5-5.5° C.

3. The method of any preceding claim, wherein the aqueous medium has a pH of 6-8, 7-8, 7.2-7.8, or 7.3-7.5.

4. The method of any preceding claim, wherein the surfactant is a non-ionic surfactant, optionally wherein the surfactant is polysorbate 20.

5. The method of any preceding claim, wherein the surfactant is present in the aqueous medium at a concentration of 4-8% w/v, or 5.5-6.5% w/v, or 5.94-6.06% w/v.

6. The method of any preceding claim, wherein the method is not performed at sink conditions, wherein sink conditions are defined as conditions wherein the equilibrium solubility of rilpivirine or a pharmaceutically acceptable salt thereof in the aqueous medium is at least 3 times higher than the concentration that would be obtained if all the rilpivirine or a pharmaceutically acceptable salt thereof from the sample dissolves in the aqueous medium.

7. The method of any preceding claim, wherein the equilibrium solubility of rilpivirine or a pharmaceutically acceptable salt thereof in the aqueous medium is at least as high as the concentration that would be obtained if all the rilpivirine or a pharmaceutically acceptable salt thereof from the sample dissolves in the aqueous medium.

8. The method of any preceding claim, wherein if all the rilpivirine or a pharmaceutically acceptable salt thereof from the sample dissolves in the aqueous medium the concentration of rilpivirine or a pharmaceutically acceptable salt thereof is about 0.015-0.025 mg/mL, or about 0.019-0.021, or about 0.020 mg/mL.

9. The method of any preceding claim, wherein the sample contains 10-30 mg, or 16-20 mg, or 17.1-18.9 mg rilpivirine or a pharmaceutically acceptable salt thereof.

10. The method of any preceding claim, wherein the volume of the aqueous medium is 500-1500 mL, or 700-1,100 mL, or about 900 mL.

11. The method of any preceding claim, wherein the aqueous medium comprises a buffer, optionally wherein the buffer is 0.05 M sodium phosphate buffer.

12. The method of any preceding claim, wherein the aqueous medium:
comprises 5.94-6.06% w/v polysorbate 20;
comprises 0.05 M sodium phosphate buffer;
has a pH of 7.3-7.5; and
is maintained at a temperature of 4.5-5.5° C.

13. The method of claim 12, wherein the sample contains 17.1-18.9 mg rilpivirine or a pharmaceutically acceptable salt thereof and the volume of the aqueous medium is about 900 mL.

14. The method of any preceding claim, comprising performing a first iteration of the method on a first sample and performing a second iteration of the method on a second sample, wherein the concentration of the surfactant in the aqueous medium in the second iteration is maintained within ±1% of the concentration of surfactant in the aqueous medium in the first iteration, the temperature of the aqueous medium in the second iteration is maintained within ±0.5° C. of the temperature of the aqueous medium in the first iteration, and the pH of the aqueous medium in the second iteration is maintained within ±0.1 of the pH of the aqueous medium in the first iteration.

15. The method of any preceding claim, wherein dispersing the sample into the aqueous medium comprises agitation, optionally using a paddle apparatus.

16. The method of any preceding claim, wherein dispersing the sample into the aqueous medium is achieved using a USP apparatus, optionally achieved using USP type 2 apparatus.

17. The method of claim 15 or 16, wherein the rotation speed of the apparatus is 10-100 rpm, or 25-75 rpm, or about 50 rpm.

18. The method of any preceding claim, wherein measuring the dissolution of the rilpivirine or a pharmaceutically acceptable salt thereof in the aqueous medium is performed as a function of time, optionally over a period of 4-8 hours, or 5-7 hours, or about 6 hours.

19. The method of any preceding claim, wherein at least 85% of the rilpivirine or a pharmaceutically acceptable salt thereof from the sample dissolves after about 6 hours.

20. The method of claim 18 or 19, wherein the method provides a measured dissolution profile of the rilpivirine or a pharmaceutically acceptable salt thereof in the aqueous medium characterized by one or more, optionally all, of features (i)-(vi):
(i) at 5 minutes, ≤30% of the rilpivirine or a pharmaceutically acceptable salt thereof dissolves;
(ii) at 10 minutes, 10-40% of the rilpivirine or a pharmaceutically acceptable salt thereof dissolves;
(iii) at 30 minutes, 39-59% of the rilpivirine or a pharmaceutically acceptable salt thereof dissolves;
(iv) at 45 minutes, 45-75% of the rilpivirine or a pharmaceutically acceptable salt thereof dissolves;
(v) at 90 minutes, 64-84% of the rilpivirine or a pharmaceutically acceptable salt thereof dissolves;
(vi) at 360 minutes, ≥80% of the rilpivirine or a pharmaceutically acceptable salt thereof dissolves.

21. The method of claim 20, wherein features (ii), (iv), and (vi) are present; or wherein features (i), (iii), (v), and (vi) are present.

22. The method of claim 18 or 19, wherein the method provides a dissolution profile of the rilpivirine or a pharmaceutically acceptable salt thereof in the aqueous medium characterized by one or more, optionally all, of features (a)-(k):
(a) about 14% of the rilpivirine or a pharmaceutically acceptable salt thereof dissolves after about 5 minutes;
(b) about 25% of the rilpivirine or a pharmaceutically acceptable salt thereof dissolves after about 10 minutes;
(c) about 34% of the rilpivirine or a pharmaceutically acceptable salt thereof dissolves after about 15 minutes;
(d) about 52% of the rilpivirine or a pharmaceutically acceptable salt thereof dissolves after about 30 minutes;
(e) about 62% of the rilpivirine or a pharmaceutically acceptable salt thereof dissolves after about 45 minutes;
(f) about 69% of the rilpivirine or a pharmaceutically acceptable salt thereof dissolves after about 60 minutes;
(g) about 77% of the rilpivirine or a pharmaceutically acceptable salt thereof dissolves after about 90 minutes;
(h) about 82% of the rilpivirine or a pharmaceutically acceptable salt thereof dissolves after about 120 minutes;
(i) about 88% of the rilpivirine or a pharmaceutically acceptable salt thereof dissolves after about 180 minutes;
(j) about 91% of the rilpivirine or a pharmaceutically acceptable salt thereof dissolves after about 240 minutes;
(k) about 94% of the rilpivirine or a pharmaceutically acceptable salt thereof dissolves after about 360 minutes.

23. The method of claim 22, wherein features (a), (d), and (j) are present; or wherein features (a), (c), (e), and (i) are present.

24. The method of any preceding claim, comprising measuring the dissolution of the rilpivirine or a pharmaceutically acceptable salt thereof in the aqueous medium at an infinity point wherein about all of the rilpivirine or a pharmaceutically acceptable salt thereof from the sample dissolves.

25. The method of claim 24, wherein the infinity point is reached by increasing the temperature of the aqueous medium from 2-15° C. to room temperature or above, optionally to about 37° C., and optionally maintaining the aqueous medium at the increased temperature for about 1 hour.

26. The method of any preceding claim, wherein measuring the dissolution of the rilpivirine or a pharmaceutically acceptable salt thereof in the aqueous medium comprises removing an aliquot from the aqueous medium, optionally filtering the aliquot, and measuring the amount of rilpivirine or a pharmaceutically acceptable salt thereof dissolved in the aliquot.

27. The method of claim 26, wherein the aliquot is filtered before measuring the amount of rilpivirine or a pharmaceutically acceptable salt thereof dissolved in the aliquot, wherein the filtering is achieved using a filter with a pore size of 0.1 μm, such as a regenerated cellulose or PVDF membrane.

28. The method of any preceding claim, wherein measuring the dissolution of the rilpivirine or a pharmaceutically acceptable salt thereof in the aqueous medium is achieved using HPLC, optionally using a gradient ultra-high performance liquid chromatographic (UHPLC) method with UV detection.

29. The method of any preceding claim, wherein the micro- or nanoparticles have a Dᵥ50 particle diameter of less than 20 μm, or less than 10 μm, or less than 2 μm.

30. The method of any preceding claim, wherein the micro- or nanoparticles have a Dᵥ90 particle diameter of from about 500 nm to about 1,600 nm, a Dᵥ50 particle diameter of from about 200 nm to about 500 nm, and a Dᵥ10 particle diameter of from about 75 nm to about 200 nm.

31. The method of any of claims 1-29, wherein the micro- or nanoparticles have a Dᵥ90 particle diameter of from about 4 μm to about 6 μm, a Dᵥ50 particle diameter of from about 1.5 μm to about 2 μm, and a Dᵥ10 particle diameter of from about 300 nm to about 500 nm.

32. The method of any preceding claim, wherein the rilpivirine or a pharmaceutically acceptable salt thereof in the form of micro- or nanoparticles has a surface modifier adsorbed to the surface thereof, optionally wherein the surface modifier is a poloxamer such as poloxamer 338.

33. The method of any preceding claim, wherein the rilpivirine or a pharmaceutically acceptable salt thereof is rilpivirine.

34. The method of any preceding claim, wherein the sample is a suspension of micro- or nanoparticles of rilpivirine or a pharmaceutically acceptable salt thereof in a pharmaceutically acceptable carrier, such as a pharmaceutically acceptable aqueous carrier.

35. The method of claim 34, wherein the suspension comprises about 300 mg/mL rilpivirine or a pharmaceutically acceptable salt thereof.

36. The method of claim 34 or 35, wherein the suspension is homogenized prior to the step of dispersing the sample into the aqueous medium.

37. The method of any of claims 34-36, wherein the suspension comprises rilpivirine or a pharmaceutically acceptable salt thereof, in particular rilpivirine, and one or more of, optionally all of, the following components:
a surface modifier, in particular poloxamer 338;
an isotonizing agent, in particular glucose monohydrate;
a buffer, in particular sodium dihydrogen phosphate;
a chelating agent, in particular citric acid monohydrate;
a pH adjusting agent, in particular sodium hydroxide; and
water, in particular water for injection.

38. The method of any preceding claim, wherein the sample is suitable for administration by intramuscular or subcutaneous injection, optionally for the long-term treatment of HIV infection in a subject infected with HIV or for the long-term prevention of HIV infection in a subject at risk of being infected by HIV.

39. The method of claim 38, wherein the long-term treatment of HIV infection in a subject infected with HIV or the long-term prevention of HIV infection in a subject at risk of being infected by HIV comprises administering rilpivirine or a pharmaceutically acceptable salt thereof in the form of micro- or nanoparticles subcutaneously or intramuscularly intermittently at a time interval in the range of about 1 month, or about 2 months, or about 3 months, or about 4 months, or about 5 months, or about 6 months.

40. A method of quality control testing a sample of rilpivirine or a pharmaceutically acceptable salt thereof, wherein the sample comprises rilpivirine or a pharmaceutically acceptable salt thereof in the form of micro- or nanoparticles, the method comprising:
performing the method of any preceding claim on the sample; and
determining based on the measured dissolution of the rilpivirine or a pharmaceutically acceptable salt thereof in the aqueous medium whether the sample has passed the quality control test.

41. The method of claim 40, comprising comparing the measured dissolution of the rilpivirine or a pharmaceutically acceptable salt thereof in the aqueous medium with one or more reference values of the dissolution of a reference sample of rilpivirine or a pharmaceutically acceptable salt thereof in the form of micro- or nanoparticles and determining, based on the comparison, whether the sample has passed the quality control test.

42. The method of claim 41, wherein the one or more reference values are for the dissolution of the reference sample in an identical aqueous medium to the aqueous medium into which the sample was dispersed, optionally wherein the dissolution of the reference sample and the dissolution of the sample are tested using an identical method.

43. A method of releasing a batch of rilpivirine or a pharmaceutically acceptable salt thereof for pharmaceutical use, the method comprising:
providing a batch of rilpivirine or a pharmaceutically acceptable salt thereof in the form of micro- or nanoparticles, optionally in a suspension;
performing the method of quality control of any of claims 40-42 on a sample taken from the batch; and
if the sample passes the quality control test, releasing the batch for pharmaceutical use.

44. The method of claim 43, wherein providing a batch of rilpivirine or a pharmaceutically acceptable salt thereof in the form of micro- or nanoparticles comprises manufacturing the batch.

45. The method of claim 43 or 44, wherein the batch contains multiple doses of rilpivirine or a pharmaceutically acceptable salt thereof formulated to be suitable for administration by intramuscular or subcutaneous injection, optionally for the long-term treatment of HIV infection in a subject infected with HIV or for the long-term prevention of HIV infection in a subject at risk of being infected by HIV.

46. The method of claim 45, wherein the long-term treatment of HIV infection in a subject infected with HIV or the long-term prevention of HIV infection in a subject at risk of being infected by HIV comprises administering rilpivirine or a pharmaceutically acceptable salt thereof in the form of micro- or nanoparticles subcutaneously or intramuscularly intermittently at a time interval in the range of about 1 month, or about 2 months, or about 3 months, or about 4 months, or about 5 months, or about 6 months.

47. The method of any of claims 43-45, wherein the batch is of an approved pharmaceutical product, such as a product approved by the FDA, EMA, and/or MHRA.

48. An aqueous medium for use in dissolution testing, the aqueous medium:
comprising 4-8% w/v, or 5.5-6.5% w/v, or 5.94-6.06% w/v of a surfactant, optionally wherein the surfactant is a non-ionic surfactant such as polysorbate 20;
comprising a buffer, such as 0.05 M sodium phosphate buffer; and
having a pH of 6-8, 7-8, 7.2-7.8, or 7.3-7.5.

49. The aqueous medium of claim 48, which is maintained at a temperature of 2-15, 3-10, 4-6, or 4.5-5.5° C.

50. The aqueous medium of claim 48 or 49, comprising dissolved rilpivirine or a pharmaceutically acceptable salt thereof.
